(19) United States
(12) Patent Application Publication
(10) Pub. No.: US 2023/0348891 A1
(43) Pub. Date: Nov. 2, 2023

(54) FUNCTIONAL SCREENING USING DROPLET-BASED MICROFLUIDICS

(71) Applicants: HIFIBIO (SHANGHAI) LIMITED, Shanghai (CN); Nankai University, Tianjin (CN)

(72) Inventors: Liang Schweizer, Cambridge, MA (US); Bingqing Shen, Shanghai (CN); Hongkai Zhang, Tianjin (CN); Yuan Wang, Tianjin (CN); Ruina Jin, Tianjin (CN); Mengshi Huang, Shanghai (CN)

(21) Appl. No.: 18/021,456

(22) PCT Filed: Aug. 20, 2021
(86) PCT No.: PCT/CN2021/113823

§ 371 Date: Feb. 15, 2023

(30) Foreign Application Priority Data
Aug. 21, 2020 (WO) PCT/CN2020/110616

(51) Int. Cl. C12N 15/10

(52) U.S. Cl. CPC C12N 15/1037; C12N 15/1086

(57) ABSTRACT

Provided is a droplet-based microfluidics platform for functional screening of interacting molecules. The platform is particularly useful for high throughput screening of interacting proteins, such as antibodies or engineered cytokines, which trigger a detectable downstream signaling event.

### BACKGROUND OF THE INVENTION

Cancer immunotherapies harness the power of the immune system to treat tumor, and has become an important part of cancer treatment. The first generations of cancer immunotherapy agents consist primarily of antagonist antibodies that block negative immune checkpoints, such as programmed cell death protein 1 (PD-1) (1-3). Nevertheless, co-stimulatory receptor agonist antibodies and bispecific T-cell engager (BiTE) antibodies are becoming increasingly important in driving anticancer immunity (4-7).

Although early attempts to develop CD28 superagonists had met with unacceptable toxic effects, the areas of co-stimulatory receptor agonist has been reignited over the last decade thanks to the substantial advances in the field of immunoncology. The co-stimulatory receptors are expressed on a number of immune cell types, including T cells, B cells and natural killer (NK) cells, as well as APCs, and engagement of these receptors can promote immune cell function, proliferation and survival. Nevertheless, there are no general rules that guide the screening of agonist antibodies. For example, a panel of antibodies that bind to the same or similar epitopes of Fas receptor led to different biological effects, with some acting as agonists while others as antagonists (4, 26). The intrinsic complexity of agonist antibody required screening as many antibodies as possible in order to maximize the chance of discovering potent agonist antibodies.

Meanwhile, bispecific T or NK cell engager (BiTE or BiKE) also hold great promise for cancer treatment, and a growing number of BiTE and BiKE are making their way through various stages of development (6). To obtain the optimal BiTE or BiKE, however, a bispecific antibody library needs to be constructed to cover the complexity of the array of tumor antigen-targeting antibodies. The difficulties arise, however, with the large number of bispecific antibodies in the library that exceeds the throughput of the existing methods.

Phage display is one of the in vitro display technology that allows one to select antibody binders from a large combinatorial library (8-13). However, analogously powerful approaches are lagging for isolating antibodies whose function goes beyond simple binding, which is the case for agonist antibody and BiTE. One underlying reason is that one has to produce and test the activity of each individual antibody, an inherently slow process that is difficult to scale up for high throughput screening. Therefore, such conventional methods don’t allow one to assay more than a few thousands antibodies at one time.

Hongkai et al. has developed an autocrine based methods for selection of functional antibodies (14, 15). In that approach, reporter cells are infected with a lentiviral antibody expression library. The cell activated by the antibody secreted by itself is sorted, and the antibody can be identified by sequencing the gene of antibody in the cell.

However, this method doesn’t allow iterative screening, and cannot be easily adapted for the assays involving more than one cell. The problem is particularly acute for screening bispecific antibodies, which need to be screened in an effector cell and target cell coculture system.

The advent of droplet microfluidics technology allows screening of antibody-secreting cells at single cell level, which could not be obtained using the population-based assays. The microfluidics droplet system can encapsulate cells in the water-in-oil droplets at the rate of thousands of droplets per second (16). Antibodies generated by the cells are contained in the droplet, enabling the maintenance of phenotype and genotype linkage in the droplet (17-19). Finally the droplets containing desirable cell can be sorted by fluorescence activated droplet sorting (FADS). Bachir et al. described application of microfluidics droplet system to screen hundreds of thousands of hybridoma cells for antibody that inhibit enzyme ACE-1 or bind to target cells (20, 21). Recently Klaus et al. simultaneously analyzing antibody secretion rate and affinity of millions of individual antibody secreting cells (22), and Annabelle et al. screened millions of plasma cells for antibodies bound to vaccine or cancer target using a sophisticated microfluidics droplet system (23).

### SUMMARY OF THE INVENTION

The invention described herein provides a droplet-based microfluidics platform for functional screening of interacting molecules. The platform is particularly useful for high throughput screening of millions of interacting proteins, such as antibodies, that trigger a detectable downstream signaling event.

For example, for antibody screening, antibody genes are delivered into cells using appropriate vectors, such as lentiviral vectors, and the resulting antibody-producing cells are co-compartmentalized with a reporter cell in droplets using microfluidics system. Droplets in which the reporter cell is activated by the co-encapsulated antibody-producing cell are sorted, and antibody-secreting cells are expanded for further rounds of selection (if desired). The enriched antibodies are identified by next generation sequencing (NGS) of antibody genes in the sorted cells.

With this approach, an anti-Her2/anti-CD3 bispecific antibody and several potent CD40 agonist antibodies were discovered, most of which were too rare (<0.02% frequency) to be discovered by using the conventional method. Results described herein demonstrates the technical capability and versatility of the platform, which may revolutionize antibody drug development.

Thus the invention provides a method of identifying an agonist or antagonist polypeptide of a biological function, the method comprising: (1) providing a plurality of nano-or pico-liter droplets, each comprising: (i) no more than one library cell that (if present) expresses or is capable of expressing a candidate agonist or antagonist polypeptide from a library of candidate agonist or antagonist polypeptides; (ii) a reporter cell that, upon contacting the agonist or antagonist polypeptide of the biological function, produces a detectable signal as a marker of said biological function; (2) maintaining the plurality of nano-or pico-liter droplets under a suitable condition to permit said agonist or antagonist polypeptide to contact the report cell to trigger the biological function, thereby producing said detectable signal; (3) isolating or enriching nano- or pico-liter droplets manifesting said detectable signal, thereby identifying the agonist or antagonist polypeptide of said biological function, within the isolated or enriched nano- or pico-liter droplets.

### BRIEF DESCRIPTION OF THE DRAWINGS

FIG. 1 is a schematic overview of one embodiment of an activity-based antibody selection method using droplet-based microfluidics. Briefly, the antibody genes were cloned into lentiviral vectors. Eukaryotic cells were infected by the lentiviral antibody library and individual transduced cells were co-encapsulated with the reporter cell into droplets using microfluidics system. The resulting emulsion was incubated off-chip overnight and injected into the sorting chip. Droplets containing antibody secreting cells and activated reporter cell were sorted. The sorted cells were cultured for the next round of selection. After multiple rounds of iteration, antibody genes were amplified from the sorted cells and analyzed by Sanger Sequencing or Next Generation Sequencing (NGS). The enriched antibodies were synthesized and recombinant antibodies were expressed and tested for function.

FIG. 2A shows an exemplary embodiment of a droplet maker microfluidics chip, which can be used to generate picoliter droplets to co-encapsulate antibody secreting cells with reporter cells.

FIG. 2B shows an exemplary embodiment of a sorting chip that can be used to collect picoliter droplets based on the intensity of fluorescence inside the droplets. The functions of various inlets is indicated, and pictures of outlets for droplet generation and sorting are shown.

FIGS. 3A-3E show a typical screening of bispecific antibodies from a spike-in library with microfluidics system.

FIG. 3A is an image of nano- or pico-liter droplets generated by microfluidic device during their 24-hr incubation at 37° C.

FIG. 3B shows the double Poisson distribution of cell number in the droplets. K562-Her2 cells were stained with CellTrace Violet and Jurkat cells were stained with CellTrace Yellow. Cell loading was evaluated by counting the labeling signals within each droplet.

FIG. 3C shows comparison of plate-based culture and droplet-based culture. K562-Her2 cells were infected with anti-Her2/anti-CD3 positive control lentivirus at low MOI. Half of the infected K562-Her2 cells were cocultured with Jurkat / pIL2-eGFP reporter cell in plate well, and the other half of infected K562-Her2 cells were coencapsulated with the reporter cell (with a mean λ of 0.5 protein-secreting K562 cell per droplet). After 16 hrs of incubation, the activation of reporter cells for both conditions were analyzed (e.g., using flow cytometry).

FIG. 3D shows representative images of the droplets after sorting for anti-Her2 / anti-CD3 bispecific antibody. K562-Her2 cells were infected with anti-Her2 / anti-CD3 bispecific antibody lentivirus library. The antibody secreting K562-Her2 cells were stained with CellTrace Violet and the Jurkat / pIL2-eGFP cells were stained with CellTrace Yellow. Individual K562-Her2 cells were coencapsulated with the Jurkat / pIL2-eGFP reporter cells into droplets. After 16 hrs of incubation, the droplets were sorted.

FIG. 3E shows activation of reporter cells by the identified bispecific antibodies. The K562-Her2 cells were cocultured with Jurkat/pIL2-eGFP reporter cells in the presence of selected bispecific antibodies. Expression of GFP by the reporter cell was analyzed by flow cytometry.

FIG. 3F shows cell-mediated cytotoxicity of BiTE1. PBMCs and SK-BR-3 cells were cocultured for 48 hrs in the presence of BiTE1 or control antibody CD3-HEL. Cell lysis was determined by measuring the release of Lactic Acid Dehydrogenase (LDH) from tumor cells.

FIGS. 3G and 3I show T cell activation in the response to BiTE. SK-BR-3 cells were cocultured with PBMCs for 48 hours in the presence of BiTE1 or a control antibody. The activation of T cells was investigated by detecting CD69 expression on T cells using flow cytometry analysis (FIG. 3G). The level of IFN-γ (FIG. 3H) and IL-2 (FIG. 3I) in culture supernatant was measured by ELISA.

FIGS. 4A and 4B show screening of CD40 agonist from a spike-in library with microfluidics system. In FIG. 4A, RFP-positive hexameric CD40L protein-secreting cells were spiked into a 10-fold excess of BFP-positive irrelevant anti-HEL antibody-secreting cells, and the mixture of cells were co-encapsulated with the reporter cells. After incubation, the droplets containing activated reporter cells were sorted based on the green fluorescence. The proportion of droplets containing RFP-positive or BFP-positive cells before and after sorting was analyzed. FIG. 4B shows bright field and fluorescence images of droplets before and after sorting.

FIGS. 5A-5F show screening of CD40 agonist antibody from the combinatorial antibody library. HEK293T cells were infected with a lentivirus antibody library and individually coencapsulated with CellTrace Yellow prestained Jurkat reporter cell and fluorescence conjugated secondary antibody in droplets. After incubation, the droplets containing secreting antibody that bind and activate the co-encapsulated reporter cell were sorted. The sorted cells were then expanded for the 2^{nd} round of selection. The enriched antibodies were identified by NGS (next generation sequencing). Specifically, FIG. 5A is a schematic of possible time traces. From left to right: droplets without reporter cells, droplets containing reporter cells but secreted antibody can’t bind to target, droplets containing reporter cells and secreted antibodies can bind to reporter cell target but have no function, droplets containing reporter cells and secreted antibodies can activate the reporter cell. FIG. 5B shows the proportions of different types of droplets for each rounds of selection that were analyzed. FIG. 5C shows bright field and fluorescence images of the sorted droplets after the second round of selection. FIG. 5D is a Bar plot for the top 20 scFv clusters and their frequencies during the selection. Besides the top 20 scFv clusters, the sum frequency of other scFvs are represented in gray at the bottom of bars. FIG. 5E shows the change of frequencies of the selected antibodies during the selection. In FIG. 5F, agonist activity of the selected antibodies was tested using the CD40 reporter cell line.

FIGS. 6A-6D show characterization of antibody in in vitro and in vivo models. FIG. 6A shows the FcγRIIB dependency of antibody C04. Jurkat / NF-κB-GFP-hCD40 reporter cells were stimulated by antibody C04 or anti-HEL control in the presence of FcγRIIB overexpressing HEK293T cells. The activation of the reporter cell line was analyzed by flow cytometry. FIG. 6B shows the activation of DCs or B cells induced by C04. Human DC cells or B cells were stimulated by C04 in the absence or presence of anti-Fc antibody. The expression of CD86 was analyzed by flow cytometry. FIG. 6C shows OVA-specific CD8⁺ T cell response induced by C04 in the CD40 / FcgR humanized mice. The transgenic mice were adoptively transfered with OVA-specific OT-I cells and treated with DEC-OVA together with C04 or isotype control antibody. Mice were euthanized for the analysis of T cells. Each circle represented an individual mouse. FIG. 6D shows antitumor effect of C04 in syngeneic mouse model. CD40 / FcgR humanized mice were s.c. engrafted with MC38 tumor cells. When MC38 tumors were established (~100 mm³), mice were treated with C04, CP-870,893 or isotype control anti-HEL antibody. The tumor volume and body weight were measured every three days until the end of the experiment. Data are represented as mean ± SEM.

FIG. 7A shows development and validation of the Jurkat / pIL2-eGFP reporter cells. The Jurkat/pIL2-eGFP reporter cells were stimulated with anti-CD3 and anti-CD28 antibody overnight. GFP fluorescence was obtained by flow cytometry. FIG. 7B shows K562-Her2 cells infected with anti-Her2×anti-CD3 BiTE lentivirus or noninfected K562-Her2 cells (control) were cocultured with Jurkat/pIL2-eGFP reporter cells in the presence or absence of anti-CD28 antibody overnight. GFP expression was analyzed by flow cytometry.

FIG. 8 shows the gating strategy for the analysis of cell viability. The droplets were first gated (gate 1) to eliminate coalesced droplets and retain only droplets of the desired size. The droplet in gate 2 defines droplet containing K562-Her2 cells; gate 3 defines the droplet containing Jurkat cells. Gate 4 defines the viable cells with low fluorescent nuclear staining, indicating the live cells after 16 hrs of incubation time.

FIG. 9A shows the gating strategies for screening of anti-Her2 / anti-CD3 bispecific antibody. The droplets were first gated to eliminate coalesced droplets and retain only droplets of the desired size. The droplets containing K562 cell were gated based on CellTrace Violet fluorescence signal (gate 1). CellTrace Yellow fluorescence signal peak showed the presence of reporter cell in the droplet (gate 2). GFP fluorescence signal peak indicated activation of the reporter cell(gate 3). Lastly colocalization ⅔ and non-colocalization ½ were used to sort droplets where GFP was from Jurkat rather than K562 (gate 4 and gate 5).

FIG. 9B shows discrimination of strong and weak anti-Her2×anti-CD3 BiTE in plate well-based or droplet-based coculture systems. K562-Her2 cells infected with anti-Her2×anti-CD3, BiTE1 or BiTE3 lentivirus were cocultured with Jurkat/pIL2-eGFP reporter cells in plate wells (left panel) or individually coencapsulated with reporter cells (right panel) and incubated overnight. The activation of reporter cells in both conditions was compared.

FIGS. 10A-10C show development and validation of Jurkat / NF-κB-GFP-hCD40 reporter cells. FIG. 10A is a schematic diagram of the hexameric CD40L. Three receptor binding domains of CD40 ligand were tandemly linked to form a trivalent protein. IgG1-Fc is then used to link two of the trivalent proteins together, creating six receptor binding domains in a single agonist. In FIG. 10B, the Jurkat / NF-κB-GFP-hCD40 reporter cells were stimulated with hexameric form of CD40L overnight. GFP fluorescence was obtained using flow cytometry. In FIG. 10C, Jurkat/NF-κB-GFP-hCD40 reporter cells were stimulated with CD40L in the presence or absence of DyLight 650 anti-Fc. GFP expression was analyzed using flow cytometry.

FIG. 11 shows the Gating strategy for the analysis of the Jurkat / NF-κB-GFP-hCD40 activation in droplet. The droplets were first gated to eliminate coalesced droplets and retain only droplets of the desired size. Two gates were assigned using drop code DY638. Gate 1 defines droplets from negative emulsion where HEL cells were encapsulated, gate 2 defines droplets from positive emulsion where CD40L cells were encapsulated. Gate 3 defines the droplet containing HEL cells according to BFP signal and gate 4 defines the CD40L cells according to RFP signal. Gate 5 defines droplets where reporter cells were activated and emitted GFP fluorescence. After 16 hrs of incubation, 24% of the CD40L cell containing droplets exhibited GFP fluorescence signal while the HEL cell and reporter cell co-encapsulating droplets showed clean background of the activation of reporter cell (0.5%).

FIG. 12 shows the gating strategies for screening of CD40 agonist antibody. The droplets were first gated to eliminate coalesced droplets and retain only droplets of the desired size. The droplets of the screening population were first gated based on the intensity of DY405. CellTrace Yellow fluorescence signal peak showed the presence of reporter cell in the droplet (gate 2). The Dylight647 fluorescence peak signal indicated binding of the secreting antibodies to CD40 on the reporter cell (gate 3) and GFP fluorescence signal peak indicated activation of the reporter cell (gate 4).

FIG. 13A shows experimental time traces recorded for droplets analyzed at ~ 900 Hz and corresponding to the examples schematized in FIG. 6A. Droplets are sorted if they display a green peak (green line, GFP signal), a red peak (red line, rabbit anti-human IgG Fc DL650 gathering around reporter cells) and a yellow peak (yellow line, reporter cells) that co-localize. Blue fluorescence (blue line) signal is used to identify droplets population.

FIG. 13B shows the Fc-dependency of the hits in droplet-based coculture systems. HEK293FT cells infected with C01 or C04 lentivirus were individually coencapsulated with reporter cells in the presence or absence of crosslinking secondary antibody.

FIGS. 14A-14C show binding of C04 to human, rhesus and cynomolgus monkey CD40. FIG. 14A shows binding of C04 to human and rhesus CD40 determined by flow cytometry. 293FT cells were transient transfected with human or rhesus CD40 and incubated with different concentration of antibody C04 and goat anti-human IgG Fc Alexa Fluor 488. The cells were analyzed by flow cytometry. FIG. 14B shows binding of C04 to cynomolgus monkey CD40 determined by SPR analysis. Anti-His antibody was immobilized on Series S CM5 chip, His-tagged cynomolgus monkey CD40 were captured by the immobilized anti-his antibody. Different concentrations of CD40 antibodies were injected through flow cells and K_{d} values were calculated using the 1:1 binding kinetics model. FIG. 14C shows binding of C04 to different tumor necrosis factor superfamily (TNFSF) receptors determined by ELISA.

### DETAILED DESCRIPTION OF THE INVENTION

### 1. Overview

The invention described herein provides an efficient technology platform to simultaneously screen binding and agonistic or antagonistic activity of interacting polypeptides, such as antibody and antigen, by combining the strength of libraries (such as those carried by a lentiviral vector system) with the high throughout capability of microfluidics droplet system.

As used herein, an “antigen” is a molecule or a portion of a molecule capable of being bound by an antibody (or antigen binding polypeptide). In general, an antigen includes epitopes consist of chemically active surface groupings of molecules, for example, amino acids or sugar side chains, and have specific three-dimensional structural characteristics as well as specific charge characteristics. Epitope, as used herein, refers to the antigenic determinant recognized by the CDRs of the antibody (or an antigen-binding portion thereof). In other words, epitope refers to that portion of any molecule capable of being recognized by, and bound by, an antibody.

The term “antibody,” as used herein, may refer to an intact immunoglobulin having two light and two heavy chains that binds to an antigen of interest, or any portion or fragment thereof that binds to the antigen of interest.

The technical capabilities of the technology platform have been demonstrated in the examples herein, which show successful screening of potent co-stimulatory receptor CD40 agonist antibodies and anti-Her2/anti-CD3 bispecific antibodies from large combinatorial antibody libraries. The streamlined technology enables efficient discovery of active antibodies that may be useful for numerous biological and therapeutic utilities, such as next generation immunotherapy to treat diseases including cancer.

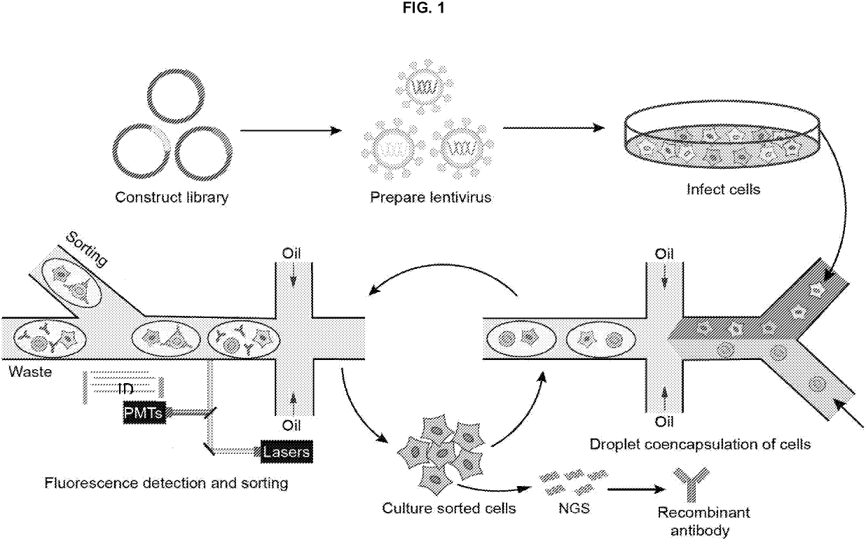

Merely to illustrate, FIG. 1 shows one exemplary embodiment of the subject function-based screening / selection process using droplet-based microfluidics.

Specifically, cells are infected by an antibody-expression library, such as a lentiviral-based antibody expression library, at low multiplicity of infection (MOI, such as 0.1 to 0.3) such that each cell is infected by no more than one lentivirus and thus expresses no more than one unique antibody. The antibody-encoding lentivirus is integrated into the cell genome in the form of provirus, resulting in cell secreting the corresponding antibody. For microfluidics system based screening, individual antibody-secreting cell is co-encapsulated with a reporter cell into a droplet by using microfluidic drop-maker. The resulting emulsion is incubated off-chip overnight, and injected into the sorting chip. The droplets containing activated reporter cells are then sorted by FADS. The cells are recovered from the sorted droplets, and the functional antibodies are identified by sequencing antibody genes in the cells.

The data presented herein illustrates the use of a droplet-based microfluidics platform for the selection of functional antibody, such as co-stimulatory receptor agonist antibody and bispecific T cell engager. The platform shared some key features with the most efficient selection methods to date such as phage display (11). First, the genotype and phenotype linkage was maintained through the whole process. Second, the product from one round can be directly amplified and used as the input of the next round of selection. Thus, multiple rounds of iteration allowed enrichment of rare hits. Compared to the conventional method to individually express and assay thousands of antibodies, the throughput of this platform increased to 10 million. The usefulness of this platform has been demonstrated in the discovery of both bispecific antibodies and agonist antibodies, which are two emerging drug modalities for cancer immunotherapy.

Indeed, the superiority of the subject platform is evidenced by the fact that the few potent CD40 agonist antibodies identified by the screen were too rare (<0.02% frequency) to be discovered by using the conventional method. Further, the ability of the platform to simultaneously screen large number of candidate bispecific antibodies provides significant opportunities to identify optimal BiTE or BiKE from large bispecific antibody libraries.

The activity-based selection method described herein also has broad applicability to the high throughput analysis of cell-cell interactions. For example, DC cells infected with lentivirus library of neoantigens can be co-encapsulated with tumor infiltrating T cells to map the pairs of cognate antigens and T cell receptors (TCR) (27).

The activity-based selection method can also be adapted to screen different types of molecules such as cytokines (28-30), such as in cytokine engineering. For example, a library of cytokine-encoding polynucleotides can be produced through, e.g., random mutagenesis and/or rational design. The library can then be expressed using the lentiviral vector of the invention in individual cells transduced by the vector, and the system and method of the invention can be used to identify engineered cytokines with altered property, such as altered binding specificity / affinity, such that they either bind to new cytokine receptors, or bind to the native receptors with fine-tuned downstream signaling and/or cellular responses (including proliferation, differentiation, activation, apoptosis, cell fate determination, etc.).

However, the invention described herein is not limited to the specific illustrative embodiments above. The methods and systems of the invention can be applied to any functional screening using a library of candidates with a reporter that generates a detectable signal which signifies a functional activity of the candidate.

Thus one aspect of the invention provides a method of identifying an agonist or antagonist polypeptide of a biological function, the method comprising: (1) providing a plurality of nano- or pico-liter droplets, each comprising: (i) no more than one library cell that (if present) expresses or is capable of expressing a candidate agonist or antagonist polypeptide from a library of candidate agonist or antagonist polypeptides; (ii) a reporter cell that, upon contacting the agonist or antagonist polypeptide of the biological function, produces a detectable signal as a marker of said biological function; (2) maintaining the plurality of nano-or pico-liter droplets under a suitable condition to permit said agonist or antagonist polypeptide to contact the report cell to trigger the biological function, thereby producing said detectable signal; (3) isolating or enriching nano- or pico-liter droplets manifesting said detectable signal, thereby identifying the agonist or antagonist polypeptide of said biological function, within the isolated or enriched nano- or pico-liter droplets.

As used herein, a “reporter cell” includes any cell that can generate a detectable signal (e.g., a light signal, such as a fluorescent signal) upon contacting a desired candidate molecule that can trigger a biological function of interest. For example, in some embodiment, the reporter cell may comprise a reporter gene encoding a fluorescent protein under the control of a promoter, which promoter is activated upon triggering the biological function of interest. Therefore, a functional antibody as a candidate molecule may bind to the surface of a reporter cell to crosslink a cell surface receptor on the reporter cell, and initiate a downstream signaling event that includes activating the promoter of the reporter gene.

In certain embodiments, the biological function is cell death, e.g., the reporter cell is dead upon triggering of the biological function. For example, the candidate molecule (e.g., functional antibody) may induce ADCC of the reporter cell, and the ability of the each candidate antibody to induce ADCC of the reporter cell may be detected by a fluorescent signal generated by the dead reporter cell.

There are many ways to detect dead cells using fluorescent signal. In one embodiment, the presence of dead cells is measured by taking advantage of the loss of membrane integrity in the dead reporter cell, and the ability of indicator molecules to partition into a compartment not achievable if the cell membrane is intact. For example, the reporter cell may encode an enzyme (such as lactate dehydrogenase or LDH) that is only leaked outside the cell into the nano- or pico-liter droplet to catalyze a chemical reaction that generates detectable color in the nano- or pico-liter droplet. For example, LDH catalyzes the conversion of pyruvate to lactate, and in the process, converts NAD⁺ to NADH, the reducing capacity of which can be used to reduce a variety of substrate molecules into products that are either colored (e.g., tetrazolium compound as the diaphorase substrate which is converted into an intensely colored formazan product), fluorescent (e.g., resazurin being converted into the fluorogenic product resorufin), or luminogenic (e.g, a pro-luciferin substrate is converted into a luciferin product that is linked to a firefly luciferase reaction to generate a luminescent signal).

In other embodiments, enzymes that do not use the NADH cycling assay chemistry can also be used as markers of dead cells, such as adenylate kinase (AK) and glyceraldehyde-3-phosphate dehydrogenase (GAPDH) that can produce ATP by providing a reaction cocktail containing the necessary ingredients to generate a cycling assay chemistry.

In another embodiment, the enzyme leaked from a dead reporter cell is a protease, such as aminopeptidase, which activity can be measured using substrates containing a short sequence of amino acids (alanine-alanine-phenylalanine) conjugated via a peptide bond to either rhodamine 110 or aminoluciferin. Enzymatic removal of the amino acids can generate free rhodamine-110 for a fluorescent assay or free aminoluciferin which can be used by firefly luciferase to generate light.

In a further embodiment, the reporter cell is pre-loaded with a measurable marker, such as pro-fluorescent Calcein-AM or radioactive ⁵¹Cr which has been used to measure ADCC. Reporter cells incubated with ⁵¹Cr take up the radioactive marker which becomes bound as protein complexes in the cytoplasm of live cells. Similarly, calcein-AM is taken up by live reporter cells where cytoplasmic esterase activity removes the AM group to generate fluorescent calcein which is retained in live cells. Upon reporter cell death and loss of membrane integrity, the fluorescent calcein or the radioactive ⁵¹Cr is released from the cytoplasm into the nano- or pico-liter droplet medium encompassing the reporter cell, where they can be identified as diffused signals (as opposed to concentrated peak signals) colocalizing with the position of the reporter cell.

In yet another embodiment, the reporter cell expresses a marker enzyme such as luciferase, which produces reduced luminescence signal when the reporter cell dies and cytoplasmic components are released outside the cell.

In another embodiment, the nano- or pico-liter droplet encompassing the reporter cell may comprise a dye (such as trypan blue or nucleic acid binding dye such as CellTox Green, YO-PRO-1, Hoechst 33342, propidium iodide, SYTOX Green nucleic acid stain, YOYO-1 Iodide, TO-PRO-3 Iodide, DRAQ7 far-red fluorescent DNA dye, and GelRed) that is not permeable through live cell membrane, but is able to permeate into a dead cell’s membrane.

As used herein, a “picoliter droplet” or “pico-liter droplet” can be produced by a microfluidic device (such as those described herein). It typically has a volume of from about 0.002-500 picoliter (pL), 0.01-400 pL, 0.1-300 pL, 1-200 pL, 10-150 pL, 50-100 pL, 50-150 pL, 50-180 pL, 50-200 pL, 60-100 pL, 60-120 pL, 60-150 pL, 60-200 pL though large (e.g., nL) or smaller (e.g., fL) volume droplets can be controllably produced by adjusting settings and/or microfluidic device architect, and are within the scope of the invention described herein. As used herein, a “nano-liter droplet” can be produced by a microfluidic device (such as those described herein). A nano-liter droplet may have a volume of from about 0.2 nanoliter (nL), 0.3 nL, 0.4 nL, 0.5 nL, 1 nL, 2 nL, 3 nL, 5 nL, 10 nL, 20 nL, to 50 nL.

Such droplets can be produced by microfluidic devices at a very high rate of between, for example, 0.1-10,000 Hz, or about 5-10,000, or more droplets per second.

As used herein, a “reporter cell” is a cell that can generate a detectable signal in response to the presence or absence of the biological function, such that the nano- or pico-liter droplet containing the reporter cell with the detectable signal can be identified or distinguished from a reporter cell without the detectable signal.

In certain embodiments, the detectable signal is a light signal, such as fluorescent light signal, that permits the nano- or pico-liter droplets containing such light signal be sorted in, for example, a fluorescence-activated cell sorting (FACS) device or an equivalent thereof.

One salient feature of the present invention is that the method can be used for functional screening of a library of molecules (e.g., polypeptides) that can be produced / expressed by library cells.

As used herein, “library cells” includes a population of cells that each produces / expresses ideally one member of a heterogeneous library of candidate molecules. “A library cell” is a cell from the library cells. Although two library cells may produce / express the same candidate molecule, collectively, the library cells together produce / express all members of the candidate library, or a substantial portion of the candidate library. In certain embodiments, each library cell produces / expresses one candidate molecule from the candidate library. In other embodiments, each library cell produces / expresses more than one candidate molecule from the candidate library.

In certain embodiments, the library cell is a eukaryotic cell, such as a plant cell, an animal cell, a mammalian cell, a unicellular organism, an insect cell (e.g., sf9), or a yeast (S. cerevisiae, S. pombe, C. albicans etc.).

In certain embodiments, the library cell is a stem cell, a cancer cell (e.g., cancer cell line or isolated cancer cell), an immune cell, a lymphocyte, a B cell, a T cell, a CD4⁺ T cell, a CD8⁺ T cell, a Treg cell, a NK cell, a NKT cell, a macrophage, a neutrophil, an eosinophil, a basophil, a monocyte, a mast cell, or a myoblast cell.

In certain embodiments, the library cell is from a relatively homogenous established cell line. In certain embodiments, the library cell is from a heterogeneous population of cells obtained from the same source, such as a tissue sample, a tumor, or an individual.

In certain embodiments, the library cell is a prokaryotic cell, such as a bacteria cell.

In certain embodiments, the library cell expresses the candidate molecule on the cell surface. In certain embodiments, the library cell produces or secrets the candidate molecule extracellularly (e.g., into the medium in which the library cell grows).

The library of candidate molecules can be any molecules that can be produced or expressed by the library cells. Exemplary candidate molecules include polypeptides or proteins, small molecules, nucleic acids, lipids, polysaccharides, etc.

In certain embodiments, the candidate molecule is an agonist of a biological function, such as a growth factor, a cytokine, a chemokine, a hormone, a stimulator of cell surface receptor such as GPCR, TCR, BCR, immune checkpoint receptor, an antibody that engages a cell surface receptor and activates the receptor, or triggers an antibody-mediated downstream event such as ADCC (antibody-dependent cell-mediated cytotoxicity) or CDC (complement-dependent cytotoxicity), etc.

In certain embodiments, the candidate molecule is an antagonist of a biological function, such as a blocking antibody that prevents the binding of a natural ligand to a cell surface receptor and inhibits the natural ligand-induce signaling.

In certain embodiments, the candidate molecules are proteins or polypeptides, including, without limitation, antibodies, bi-specific antibodies, tri-specific antibodies, a heavy chain of an antibody, a light chain of an antibody, a functional portion of an antibody, an antigen-binding portion / fragment of an antibody (including antibodies or antigen-binding fragment thereof having similar CDR sequence except for random mutations in the CDR sequences for affinity maturation), a cytokine, or a chemokine, or a derivative thereof.

The library of cells can each produce / express a candidate member from a library through, for example, introducing an expression vector into the library cells. Any expression vectors can be used with the method of the invention.

In certain embodiments, the expression vector is a viral vector, such as a retroviral vector, a sindbis viral vector, a lentiviral vector, an adenoviral vector, an AAV vector, a plant viral vector (such as tobacco mosaic virus or TMV vector), or a hybrid thereof. In certain embodiments, the expression vector is a plasmid that can be introduced into the library cells by transfection. The library cells can be infected or transfected by the expression vectors, each encoding a unique candidate molecule, such that expression or production of the candidate molecules by the library cells can be screened using the method of the invention.

In certain embodiments, the library constructed into the viral vector (e.g., lentiviral vector) originates from a larger library having more non-redundant library members, such as (an scFv) phage display library having more than 10⁸, 10⁹, 10¹⁰, 10¹¹, 10¹² or more members. The phage display library can be pre-panned against an antigen of interest to enrich for members having a threshold binding affinity for the antigen, before the enriched members are introduced into the viral vector for use in the methods of the invention.

In certain embodiments, the expression or production of the candidate molecule can be controlled (e.g., induced or suppressed). This can be advantageous since it allows more precise control about the timing and/or extent of expression / production of the candidate molecule to be screened, and/or the timing of detection of expression / production by the reporter cell.

Thus, in certain embodiments, the expression of the candidate agonist or antagonist polypeptide from the library is under the control of an inducible promoter inducible by an activator or an activating condition.

In certain embodiments, the inducible promoter is a positive inducible promoter, and wherein an activator for said positive inducible promoter is introduced into the plurality of nano- or pico-liter droplets subsequent to the formation of the plurality of nano- or pico-liter droplets.

A “positive inducible promoter” is one that inactive in the OFF state, because, for example, an activator protein of the promoter, though maybe present, cannot bind to the promoter. Only after an inducer or activator binds to the activator protein can the activator protein be able to bind to the positive inducible promoter, thus turning it ON and initiating transcription from the promoter. In certain embodiments, the activator is a small molecule activator.

In certain embodiments, the positive inducible promoter is a Tet-ON promoter, and wherein the activator is tetracycline or a derivative thereof capable of binding to activator rtTA (reverse tetracycline-controlled transactivator).

In certain embodiments, the positive inducible promoter is an alcohol-regulated promoter (such as the AlcA promoter), and wherein the activator is AlcR or AlcA.

In certain embodiments, the positive inducible promoter is a steroid-regulated promoter (such as the LexA promoter), and wherein the activator is XVE.

In certain embodiments, the inducible promoter is a negative inducible promoter, and wherein an activator for the negative inducible promoter is introduced into the plurality of nano- or pico-liter droplets subsequent to the formation the plurality of nano- or pico-liter droplets.

A “negative inducible promoter” is one that is inactive in the OFF state because a bound repressor protein actively prevents transcription. Once an inducer binds the repressor protein, the repressor protein is removed from the DNA. With the repressor protein absent, transcription is turned ON.

In certain embodiments, the negative inducible promoter is a pLac promoter, and wherein the activator is lactose or a derivative thereof (such as IPTG) capable of binding to lac repressor (lacI protein).

In certain embodiments, the negative inducible promoter is a pBad promoter, and wherein the activator is arabinose capable of binding to AraC.

In certain embodiments, the inducible promoter is a temperature sensitive promoter, and the expression of the candidate agonist or antagonist polypeptide from the library is under the control of a temperature change as the activating condition that activates the inducible promoter.

Temperature sensitive expression systems are typically less leaky, and can have near-zero expression at regular temperatures but can be induced by heat or cold exposure. Examples include the heat shock-inducible Hsp70 or Hsp90-derived promoters, in which a gene of interest (such as the candidate agonist or antagonist in the candidate library) is only expressed following exposure to a brief heat shock. In the case of Hsp70, the heat shock releases heat shock factor 1 (HSF-1), which subsequently binds to heat shock elements in the promoter, thereby activating transcription.

In certain embodiments, the inducible promoter is a light inducible promoter (such as the FixK2 promoter), and the expression of the candidate agonist or antagonist polypeptide from the library is under the control of a light signal as the activating condition that activates the light inducible promoter.

For example, an exemplary light inducible promoter can be regulated by the blue-light sensing protein YFI. When light is absent, YFI phosphorylates FixJ, which binds to the FixK2 light inducible promoter to induce transcription. When light is present, YFI is inactive, preventing transcription from the light inducible promoter.

In certain embodiments, the inducible promoter is a pH sensitive promoter, and the expression of the candidate agonist or antagonist polypeptide from the library is under the control of a pH change as the activating condition that activates the inducible promoter.

For example, the E. coli asr (acid shock RNA) gene encodes small RNA that is inducible by low external pH, and asr gene may be regulated by the two component system PhoBR2. In this two-component system, the protons from the environment (H⁺) activate the sensory part (PhoR-) of the two-component system, which then transduces the signal to the activator protein PhoB, which can bind promoter of asr to initiate transcription. The promoter region of asr contains a sequence similar to the pho-box, which is a consensus sequence able to bind to PhoB.

In certain embodiments, the pH sensitive promoter is Pasr or PgadA.

In any of the embodiments herein, one or more additional components, such as an activator, an inducer, an additional cell, or a bolus of liquid value with acid or base used for pH change may need to be introduced into the nano- or pico-liter droplets, which can be accomplished by any of many means known in the art, such as by injection or fusion.

For example, the one or more additional components can be directly injected into each of the plurality of nano- or pico-liter droplets in a high throughput fashion. Alternatively, the one or more additional components can be encapsulated in their own nano- or pico-liter droplets that can be similarly generated by microfluidic devices, and such nano- or pico-liter droplet having the one or more additional components can be used to each of the plurality of nano- or pico-liter droplets containing the library cell and/or the reporter cell. In certain embodiments, the fusion is mediated by geometrical constraint, mechanical force, surface property change, electrical, laser, acoustic force, or any art-recognized methods. See, for example, Ahn et al., Appl. Phys. Lett. 88:3, 2006; Priest et al., Appl. Phys. Lett. 89:134101, 2006; and Songet al., Appl. Phys. Lett. 83: 4664, 2003, all incorporated herein by reference.

In certain embodiments, prior to step (1), a first plurality of nano- or pico-liter droplets each comprising the no more than one library cell have been maintained under a pre-determined condition for a pre-determined period of time to allow said candidate agonist or antagonist polypeptide to express, before said reporter cell is introduced into each said first plurality of nano- or pico-liter droplets to provide the plurality of nano- or pico-liter droplets in step (1). This may be advantageous because expression / production of the candidate molecule by the library cells can be separately controlled, either through induction, suppression, and/or time period for expression, until a desired concentration of the candidate molecule in the nano- or pico-liter droplets is reached, before the reporter cell is introduced into the nano- or pico-liter droplet for detection.

Substantially the same means can be used to introduce the reporter cells into each of the nano- or pico-liter droplets containing the library cells, including by injection or fusion. The fusion may be mediated by geometrical constraint, mechanical force, surface property change, electrical, laser, acoustic force, or any art-recognized methods.

In certain embodiments, the library cell that expresses or is capable of expressing a candidate agonist or antagonist polypeptide is pre-stained with a first tracking signal (e.g., CellTrace Violet), and said reporter cell is pre-stained with a second, different, tracking signal (e.g., CellTrace Yellow) prior to step (1), and wherein step (3) is carried out by retrieving nano- or pico-liter droplets that: (I) contain both the first and the second tracking signals; (II) produce said detectable signal (e.g., GFP) after step (2); and, (III) exhibit colocalization of the second (reporter cell) tracking signal and the detectable signal.

In certain embodiments, steps (1)-(3) are repeated more than once, using the library cells isolated or enriched in step (3) of a previous repeat. That is, after the detection and sorting of positive nano- or pico-liter droplets containing the detectable signal that signifies the presence or absence of the biological function of interest, the positive nano- or pico-liter droplets can be collected, optionally with the library cells within these positive nano- or pico-liter droplets pooled, expanded and/or further cultured, before the pooled, expended and/or cultured library cells from the first round is again encapsulated with the same or a different reporter cell in a new plurality of nano- or pico-liter droplets for a further round of screening, for the same biological function or for a different biological function.

After one or more rounds of screening, the nano- or pico-liter droplets having the positive detectable signal can be collected and the library cells within obtained, in order to determine the identity of the candidate library member that gives right to the detectable biological function, then identifying the candidate molecule as the agonist or antagonist, as the case may be.

The methods of the invention can be implemented in numerous specific settings to screen large number of candidate molecules that may exhibit a desired biological function. In certain embodiments, the library of candidate molecules has more than 10³, 10⁴, 10⁵, 10⁶, 10⁷, 10⁸, 10⁹, 10¹⁰, 10¹¹ or more non-redundant members (e.g., millions of non-redundant antibody coding sequences). In certain embodiments, the plurality of nano- or pico-liter droplets comprises more than 10³, 10⁴, 10⁵, 10⁶, 10⁷, 10⁸, 10⁹, 10¹⁰, 10¹¹ droplets.

The method of the invention can be used in numerous functional screenings.

For example, in one embodiment, the agonist or antagonist polypeptide is a bispecific T cell engager (BiTE) comprising a first antigen-binding fragment (such as a 1^{st} scFv) specific for a first antigen fused to a second antigen-binding fragment (such as a 2^{nd} scFv) specific for a second antigen.

In certain embodiments, the first antigen is a T cell antigen (such as CD3), and the second antigen is a surface antigen on a target cell (such as a cancer antigen (e.g., HER2) on a target cancer cell).

In certain embodiments, in each nano- or pico-liter droplet having said one library cell that expresses or is capable of expressing said candidate agonist or antagonist polypeptide, said candidate agonist or antagonist polypeptide is a BiTE from a library of candidate BiTEs each encoded by a lentiviral vector from a lentiviral vector library encoding said library of candidate BiTEs, and wherein said one cell is the target cell that expresses said target cancer antigen (e.g., HER2).

In certain embodiments, the reporter cell is a T cell-derived cell line (e.g., Jurkat cell) that produces a fluorescent protein (e.g., GFP), the transcription of which encoding RNA is under the transcriptional control of a promoter (e.g., IL-2 promoter) activated by T cell activation upon binding of the BiTE to the TCR of the reporter cell and the target cancer antigen on the target cell.

In certain embodiments, the library of candidate agonist or antagonist polypeptides is a library of candidate BiTEs encoded by a lentiviral vector-based library, and wherein coding sequence for each of said second antigen-binding fragment (such as a 2^{nd} scFv) specific for the second antigen has been pre-selected from a phage display library based on biopanning against said second antigen.

In certain embodiments, the complexity of the phage display library is about 10¹⁰ members, and wherein the complexity of the library of candidate BiTEs with respect to the second antigen-binding fragment is 10⁵ members.

In certain embodiments, the one library cell that expresses or is capable of expressing the candidate agonist or antagonist polypeptide (BiTE) is produced by infection at low MOI, by a lentiviral vector-based library encoding said library of candidate agonist or antagonist polypeptides (BiTEs), to ensure that each cell produces no more than one type of the candidate agonist or antagonist polypeptide (BiTE).

In certain other embodiments, the agonist or antagonist polypeptide is an agonist or antagonist antibody or an antigen-binding fragment thereof specific for a cell surface receptor (e.g., CD40) that triggers said biological function.

In certain embodiments, in each nano- or pico-liter droplet having said one cell that expresses or is capable of expressing said candidate agonist or antagonist polypeptide, said candidate agonist or antagonist polypeptide is an scFv-IgG1 Fc fusion from a library of candidate scFv-IgG1 Fc fusions each encoded by a lentiviral vector from a lentiviral vector library encoding said library of candidate scFv-IgG1 Fc fusions, optionally, wherein said cell surface receptor is CD40 and wherein said biological function is NFκB signaling.

In certain embodiments, the reporter cell is a cell line (e.g., Jurkat cell) that produces a fluorescent protein (e.g., GFP), the transcription of which encoding RNA is under the transcriptional control of a promoter (e.g., NFκB promoter) activated by activation of said cell surface receptor (e.g., CD40) upon binding of the agonist antibody or antigen-binding fragment thereof to the cell surface receptor (e.g., CD40) of the reporter cell.

In certain embodiments, the coding sequence for each of said scFv in said library of candidate scFv-IgG1 Fc fusions has been pre-selected from a phage display library based on biopanning against said cell surface receptor (e.g., CD40).

In certain embodiments, the complexity of the phage display library is about 10¹⁰ members, and wherein the complexity of the library of candidate scFv-IgG1 Fc fusions with respect to the second antigen-binding fragment is 10⁵ members.

In certain embodiments, the one library cell that expresses or is capable of expressing the candidate agonist or antagonist polypeptide is produced by infection at low MOI, by a lentiviral vector-based library encoding said library of candidate agonist or antagonist polypeptides, to ensure that each cell produces no more than one type of the candidate agonist or antagonist polypeptide.

In certain embodiments, a secondary antibody specific for said candidate agonist or antagonist polypeptide is labeled with a first tracking signal (e.g., Dylight647-conjugated) and co-encapsulated into the nano- or pico-liter droplets in step (1), and said reporter cell is pre-stained with a second, different, tracking signal (e.g., CellTrace Yellow) prior to step (1), and wherein step (3) is carried out by retrieving nano- or pico-liter droplets that: (I) contain both the first and the second tracking signals; (II) produce said detectable signal (e.g., GFP) after step (2); and, (III) exhibit colocalization of the first (CD40 agonist antibodies) and the second (reporter cell) tracking signals and the detectable signal.

In yet another embodiment, the agonist or antagonist polypeptide is an engineered or modified cytokine for a cytokine receptor that triggers said biological function.

In certain embodiments, the engineered or modified cytokine has altered specificity and/or affinity towards the cytokine receptor compared to the cognate wild-type cytokine.

In certain embodiments, the engineered or modified cytokine binds to and activates a cytokine receptor to which a cognate wild-type cytokine does not bind.

In certain embodiments, the engineered or modified cytokine stimulates or inhibits a downstream signaling pathway that is not stimulated by a cognate wild-type cytokine.

In certain embodiments, the engineered or modified cytokine commits a cell to a differentiation, proliferation, activation, and/or apoptotic process that is not stimulated or inhibited by a cognate wild-type cytokine, or is not stimulated or inhibited by the cognate wild-type cytokine to the same degree.

In certain embodiments, the method of the invention can be used to screen for agonist or antagonist polypeptide (e.g., antibody) that can induce ADCC against an antigen expressed on the reporter cell. For example, a library of antibodies can be expressed by the library cells, each can be encapsulated with a reporter cell in a nano- or pico-loiter droplet using the method of the invention. When / if the antibody recognizes the the antigen expressed on the reporter cell, an effector cell (such as NK cell) also encapsulated in the nano- or pico-liter droplet can kill the reporter cell. The presence of the dead reporter cell can be detected by a light-generating reaction using any of the suitable methods described herein (such as generating a detectable fluorescent signal by an LDH enzyme leaked outside the reporter cell). The NK / effector cell can be introduced into the nano- or pico-liter droplet using injection or fusion as described herein, or be included in the initial nano- or pico-liter droplet with the library cell and the reporter cell.

For antagonistic assay, the library cell expresses a candidate polypeptide that can potential block effector / NK cell-mediated ADCC on the target cell, and the absence / reduction of killing of the reporter cell can be measured by the fluorescent signal generated by the reporter cell. The NK / effector cell can be introduced into the nano- or pico-leter droplet at a later time through inhection or fusion to permit accumulation of the antagonistic polypeptide inside the droplet to reach a critical concentration.

In certain embodiments, the method of the invention can be used to screen for agonist or antagonist polypeptide (e.g., antibody) that can induce CDC against an antigen expressed on the reporter cell. For example, the library cell may express a candidate antibody recognizing an antigen expressed on the surface of the reporter cell, and the complement pathway components required for CDC can either be provided in the medium for encapsulating cells into the nano- or pico-liter droplets or expressed by the library or reporter cells. Dead reporter cells due to CDC can be detected using any of the methods described herein for generating fluorescent or other detectable signals in dead cells.

In yet other embodiments, the method of the invention can be used for high throughput screening of cell-cell interaction, wherein, for example, dendritic cells or other antigen-presenting cells (APCs) can be infected by a library of neoantigens in lentiviral or other suitable vectors. Such APCs are then co-encapsulated with tumor infiltrating T cells (TILs) as reporter cells capable of generating fluorescent signals upon TCR activation, in order to map the pairs of cognate antigens and T cell receptors (TCR).

In certain embodiments, step (1) is carried out with a nano- or pico-liter droplet-producing microfluidic device comprising: (a) a first inlet for an oil to form a continuous oil phase; (b) a second inlet for an aqueous suspension of a population of said reporter cell; (c) a third inlet for an aqueous suspension of a population of said cell that expresses or is capable of expressing a candidate agonist polypeptide; (d) an outlet for retrieving said nano- or pico-liter droplets dispersed in said continuous oil phase; and, (e) a junction area where the first, the second, and the third inlets converge to form nano- or pico-liter droplets in the continuous oil phase before exiting through the outlet.

In certain embodiments, step (3) is carried out in a nano- or pico-liter droplet-sorting microfluidic device comprising: (A) a first inlet of spacing oil and a second inlet of bias oil; (B) a third inlet of retrieved nano- or pico-liter droplets after step (2); (C) a first outlet for retrieving nano- or pico-liter droplets manifesting said detectable signal; (D) a second outlet for collecting waste not retrieved by the first outlet; (E) a sorting actuator that directs a passing nano- or pico-liter droplet to the first outlet when the passing nano- or pico-liter droplet manifests the detectable signal, and directs the passing nano- or pico-liter droplet to the second outlet otherwise; and, (F) a junction area where the first, the second, and the third inlets converge to form a stream of passing nano- or pico-liter droplets before the sorting actuator, and where the first and second outlets diverge to separate said nano- or pico-liter droplets manifesting said detectable signal from the waste.

In certain embodiments, the agonist or antagonist polypeptide is identified through identifying the coding sequence thereof from said cell that expresses or is capable of expressing said agonist or antagonist polypeptide retrieved from said nano- or pico-liter droplets manifesting said detectable signal. In certain embodiments, the coding sequence is identified through high throughput sequencing, such as next-generation sequencing (NGS) of the antibody encoding sequences from the isolated cells.

In certain embodiments, said cell that expresses or is capable of expressing said agonist or antagonist polypeptide can be retrieved for further processing to identify the coding sequence(s) of the polypeptide of interest. Such further processing may include the droplet-based transcriptome analysis of said cell, e.g., as described in PCT international patent application published as WO2017/097939 and Gérard, A. et al. (2020). High-throughput single-cell activity-based screening and sequencing of antibodies using droplet microfluidics. Nature Biotechnology, 38, 715-721.

In certain embodiments, the method further comprises verifying that said agonist or antagonist polypeptide leads to said biological function, including activation of said biological function by said agonist or antagonist polypeptide in a manner depending on binding by said agonist or antagonist polypeptide.

### 2. Design of the Microfluidics Platform

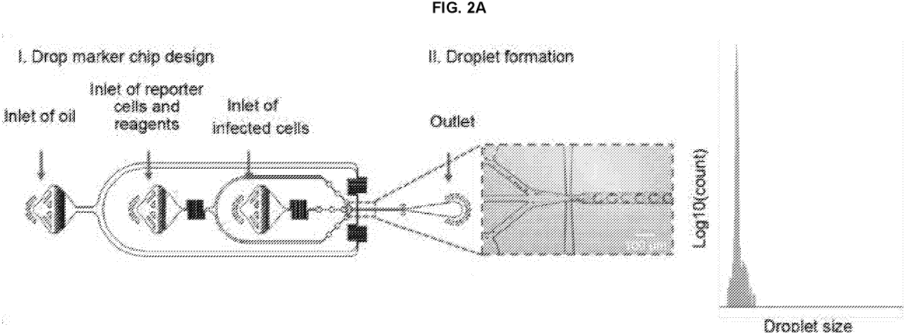

Two microfluidics devices are particularly useful in the methods of the invention: (i) a droplet formation microfluidic device (or “droplet formation device”) that compartmentalizes the library cells (e.g., library of lentivirus infected cells) with the reporter cells and/or detection reagents; and (ii) a droplet sorting device that sorts droplets based on reporter cell activation and receptor binding signals using a chosen mechanism, such as surface acoustic wave based sorter (see FIGS. 2A-2C) (24).

In certain embodiments, droplet formation and fluorescence analysis are performed on a dedicated droplet microfluidics platform reported previously in Gerard A. et al (23), incorporated herein by reference. Briefly, the microfluidic chips can be fabricated in polydimethylsiloxane (PDMS) polymer (Sylgard 184 elastomer kit; Dow Corning Corp) using the standard soft lithography as described (31). Masters are made using one layer of SU-8 photoresist (MicroChem). The depth of the two devices is 40+/-1 µm to allow the droplet generating or flowing in a monolayer format. For sorting device, the PDMS is bonded to a piezoelectric substrate (Y128-cut Lithium niobate wafer) where an golden interdigital electrode is patterned with standard lift-off technology and aligned with the fluidic channel above. Microfluidics devices are treated before use with 1% v/v ¹H,¹H,²H,²H-perfluorodecyltrichlorosilane (Alfa Aesar) in Novec HFE7500 fluorinated oil (3 M) to prevent droplets wetting the channel walls.

However, the microfluidic devices suitable for use with the methods of the invention is not so limited.

In certain embodiments, the droplet formation device comprises a T-junction or a flow-focusing device or portion or mechanism that forms monodisperse (e.g., <1-3% disperity) droplets at rates up to around 10 kHz. See, for example, Thorsen et al., Phys. Rev. Lett. 86:4163, 2001; Nisisako et al., Lab Chip 2:24, 2002; and Link et al., Phys. Rev. Lett. 92:054503, 2004, incorporated herein by reference.

In certain embodiments, the droplet formation device utilizes jetting to form droplets at a rate of 10′s of kHz. See, for example, Utada et al., Phys. Rev. Lett. 99: 094502, 2007; Utada et al., Phys. Rev. Lett. 100:014502, 2008; and Humphry et al., Phys. Rev. E 79:056310, 2009, incorporated herein by reference.

In certain embodiments, the droplet formation device utilizes membranes or sieves to produce multiple droplets simultaneously. Such membrane emulsification devices produce droplets by dispersing one fluid into a continuous phase through a membrane or sieve, essentially forming an array of T-junctions to increase rate of droplet formation by orders of magnitude. See Sugiura et al., Langmuir 17:5562, 2001, incorporated herein by reference.

In certain embodiments, the droplet formation device is an on-demand droplet formation device which ensures that only perfectly monodisperse droplets enter the device. Such on-demand droplet formation devices utilize sudden changes in applied pressure, in combination with narrow channels at the flow-focusing section of the device and rapid withdrawal of the water flow from a budding droplet. See Lorenz et al., Anal. Chem. 78:6433, 2006; and He et al., Anal. Chem. 77:1539, 2005, incorporated herein by reference.

In certain embodiments, the droplet formation device comprises an on-chip piezo-electric actuator to provide previse control over droplet formation.

In certain embodiments, the droplet formation device comprises a heating element that can, for example, provide a heat change for inducible expression of genes in the library cells under the control of a heat-sensitive promoter. The heating element can also provide additional control over droplet size, independent of device geometry and flow rates, by changing interfacial tension and viscosity to modulate droplet diameter formed in a flow focusing device. See Nguyen et al., Appl. Phys. Lett. 91:084102, 2007, and Tan et al., J. Phys. D 41:165501, 2008, incorporated herein by reference.

In certain embodiments, the droplet formation device comprises a cell-triggered Rayleigh-Plateau instability in a flow-focusing geometry to maximize single-cell compartmentalization during jet break-up. In this embodiments, up to about 70-80 % of the injected cell population is encapsulated into drops containing one and only one cell, with <1 % contamination by empty droplets. Chabert et al., Proc. Natl. Acad. Sci. USA 105:3191, 2008; and Edd et al., Lab Chip 8:1262, 2008, incorporated herein by reference.

Although droplet formation does not necessarily require surfactants, in certain embodiment, the droplet formation device of the invention utilizes a stabilizing agent to prevent or inhibit rapid coalescence of the formed droplets inside the droplet formation devices. In certain embodiments, the continuous phase is a hydrocarbon or fluorocarbon oils for forming water-in-oil emulsion. In certain embodiments, the continuous phase is a mineral oil (including fluorinated oil), optionally comprising commercially available surfactants such as Span 80, Abil EM, and Krytox (DuPont) (which contains a perfluoropolyether (PFPE) tail and a carboxylic acid hydrophilic head group) for fluorous oil continuous phases. In certain embodiments, the continuous phase comprises a fluorinated oil, and optionally comprises a fluorinated surfactant.

In certain embodiments, the continuous phase comprises a oligoethylene glycol (OEG)-terminated surfactant. In certain embodiments, the OEG surfactant is an OEG fluorinated surfactant.

In certain embodiments, the surfactant comprises a hydrophilic head group, such as a PFPE surfactant. In certain embodiments, the surfactant comprises an ammonium salt of carboxy-PFPE and/or poly-L-lysine. In certain embodiments, the surfactant comprises polyethyleneglycol (PEG) and dimorpholinophosphate (DMP), or DMP-PFPE surfactant. Such surfactant provides excellent stability in addition to biocompatibility, in that with DMP-PFPE surfactant in fluorinated oil, cells encapsulated in droplets on a microfluidic chip as emulsion can be stored for up to 14 days off chip, and then re-injected into a microfluidic device with minimal coalescence (<10 % after 14 days).

In certain embodiments, the surfactant has a critical micelle concentration (CMC) of 10⁻⁴ mol L⁻¹ or greater.

In certain embodiments, the surfactant is synthesized from 600 g/mol PEG and 6000 g/mol PFPE.

In certain embodiments, the surfactant facilitates droplet fusion through controlling droplet surface chemistry by tuning the surfactant concentration and surfactant accumulation time on chip (see Mazutis et al., Lab Chip 9:2665, 2009, incorporated herein by reference).

In certain embodiments, the methods of the invention call for droplet injection to introduce additional reagents and/or cells into pre-formed nano- or pico-liter droplets, which can be achieved using a varieties of mechanisms.

In certain embodiments, the droplet formation device comprises a picoinjector, a robust device to add controlled volumes of reagent using electro-microfluidics at kHz rates. It can also perform multiple injections for serial and combinatorial additions. See Abate et al., Proc Natl Acad Sci USA 107(45): 19163-19166. 2010, incorporated herein by reference.

In certain embodiments, the additional reagents and/or cells are introduced into the nano- or pico-liter droplets through printed droplet microfluidics (PDM), which can be used to construct defined reactions with chemicals and cells incubated under air on an open array. See Siltanen et al., Sci Rep 8:7913, 2018, incorporated herein by reference.

In certain embodiments, the methods of the invention call for droplet fusion, which can be achieved using a varieties of mechanisms.

In certain embodiments, the droplet formation device is a passive fusion devices which relies on channel properties and/or surface properties of the channels to induce droplet coalescence. For example, in-channel droplet fusion is initiated when two or more droplets are brought into close contact by draining of the continuous phase, and imbalances in surface tension leads to droplets coalescence (see Tan et al, Lab Chip 4:292, 2004, incorporated herein by reference).

In certain embodiments, the droplet formation device facilitates the fusion or more than two droplets by controlling surface energy patterns inside microfluidic channels. For example, the channels can be designed to disrupt the flow of the droplets, trapping them to the pattern and only releasing them after coalescence. Varying channel and pattern dimensions as well as fluid flows provide full control over droplet fusion, allowing the incorporation of several components into a single, large droplet by coalescence of multiple droplets. See Fidalgo et al., Lab Chip 7: 984, 2007, incorporated herein by reference).

In certain embodiments, the droplet formation device facilitates in-channel fusion by incorporating rows of pillars within the channels that act as passive merging elements. The pillars trap droplets while allowing the continuous phase to drain when a second droplet enters the pillar area. The fusion process is independent of the inter-droplet separation but is rather controlled by droplet size. Moreover, the number of droplets that can be fused at any time can also be controlled by the mass flow rate and volume ratio between the droplets and the merging chamber. See Niu et al, Lab Chip 8:1837, 2008.

In certain embodiments, the droplet formation device facilitates fusion by relying on transient states in the stabilization of the droplet interface by surfactant, coupled to a proper geometrical design of a coalescence module, to induce the selective fusion of a droplet stabilized by surfactant (re-injected) with a droplet which is not fully stabilized (generated on-chip). See Mazutis et al., Lab Chip 9(18): 2665-72, 2009, incorporated herein by reference.

In certain embodiments, the droplet formation device comprises an active fusion mechanism that can be controlled (e.g., be switched on or off). The device has the ability for achieving accurate droplet synchronization to put the droplets in very close proximity. See Thiam et al., Phys. Rev. Lett. 102:188304, 2009.

In certain embodiments, the active fusion mechanism relies on an external trigger to induce coalescence.

In certain embodiments, the external trigger is a strong electric field (see Link et al, Angew. Chem. 118:2618, 2006 and Mazutis et al, Lab Chip 9:2902, 2009, incorporated herein by reference). In certain embodiments, a smaller droplet is fused with a larger droplet because smaller drops move faster due to the Poiseuille flow, thus ensuring that they are in contact in the region where they are coalesced with an electric field.

In certain embodiments, the external trigger is an alternating current (AC) field (see Chabert et al., https://doi.org/10.1002/elps.200500109, 2005).

In certain embodiments, the external trigger is a magnetic field. See Varma et al., Sci Rep 6:37671, 2016, incorporated herein by reference. Specifically, a uniform magnetic field is used to induce merging of ferrofluid based droplets, and control of droplet velocity and merging can be achieved through uniform magnetic field and flow rate ratio.

In certain embodiments, the external trigger is a localized heating induced by laser pulse (see Baroud et al., Lab Chip 7:1029, 2007, incorporated herein by reference).

In certain embodiments, the droplet formation device hydrodynamically couples the two droplet-formation channels to ensure perfectly alternating droplet sequences. See Frenz et al., Langmuir 24:12073, 2008, incorporated herein by reference. For example, hydrodynamic resistance can be exploited to synchronize droplets in two parallel channels by using passive devices such as loops or ladders to ensure perfect alternation of droplets into two channels at a T-junction, thus leading to symmetric splitting of droplet trains.

In certain embodiments, the droplet formation device facilitates droplet fusion with a surface through a chemistrode device. See Liu et al, Langmuir 25:2854, 2009, incorporated herein by reference.

Additional fusion mechanisms that can be used with the methods of the invention are known in the art, such as those described in Xu et al., Droplet Coalescence in Microfluidic Systems (core.ac.uk/download/pdf/143869049.pdf, incorporated herein by reference).

In certain embodiments, the library cells can be incubated in the nano- or pico-liter droplets for a pre-determined time with the reporter cell, or before being in contact with the reporter cell in the nano- or pico-liter droplet.

In certain embodiments, the pre-determined time is minutes to hours, 1-5 hours, 10-24 hrs, 1-5 days, 5-10 days, or up to 2-3 weeks. The droplets can be incubated in the microfluidic device (e.g., in a storage reservoir) or collected from the device after the droplet formation, and incubated under a different condition (e.g., temperature).

In certain embodiments, the droplet sorting device is an integral part of the droplet formation device.

In certain embodiments, the droplet sorting device is separate from the droplet formation device.

In certain embodiments, the sorting device utilizes electric field to isolate droplets having the desired detectable signal. See Ahn et al., Appl. Phys. Lett. 88: 024104, 2006, incorporated herein by reference.

In certain embodiments, the sorting device utilizes surface acoustic wave to isolate droplets having the desired detectable signal. See Franke et al., Lab Chip 9:2625, 2009, incorporated herein by reference. Surface acoustic wave devices are capable of deflecting droplets or particles by locally compressing fluids.

In certain embodiments, the sorting device utilizes magnetic fields to isolate droplets loaded with magnetic particles and have the desired detectable signal. See Zhang et al., Lab Chip 9:2992, 2009, incorporated herein by reference.

In certain embodiments, the sorting device utilizes laser-induced localized heating to isolate droplets having the desired detectable signal. See Baroud et al., Phys. Rev. E 75:046302, 2007, incorporated herein by reference. An added benefit of integrating a microheater element in the device offers precise control over droplet motion (leading to both splitting and sorting of droplets) at Y-junctions through changes in fluidic resistance and the thermocapillarity in one of the branches.

In certain embodiments, the detectable signal is a fluorescent light. The electric signal obtained from fluorescence light detected at a photon multiplier tube (PMT) can be used to trigger further droplet manipulations in, for example, in the sorting device in a manner similar to a flow cytometer. The fluorescence activated sorting, or FADS (fluorescence-activated droplet sorting), combines fluorescence intensity detection with selective emulsion separation to extract target droplets into a continuous aqueous stream for collection or further manipulation.

Unless specifically disclaimed or improper, any one embodiment of the invention can be combined with one or more additional embodiments of the invention.

### EXAMPLES

### Example 1 Development and Optimization of Droplet-Based Assay

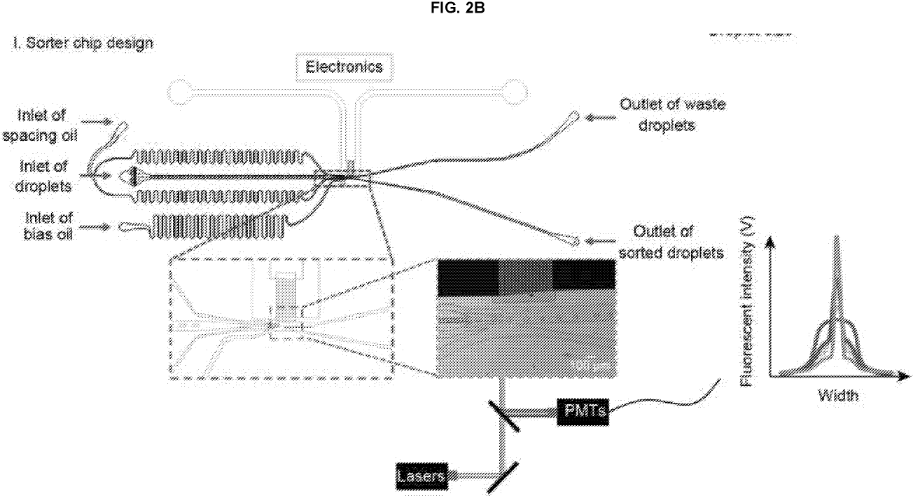

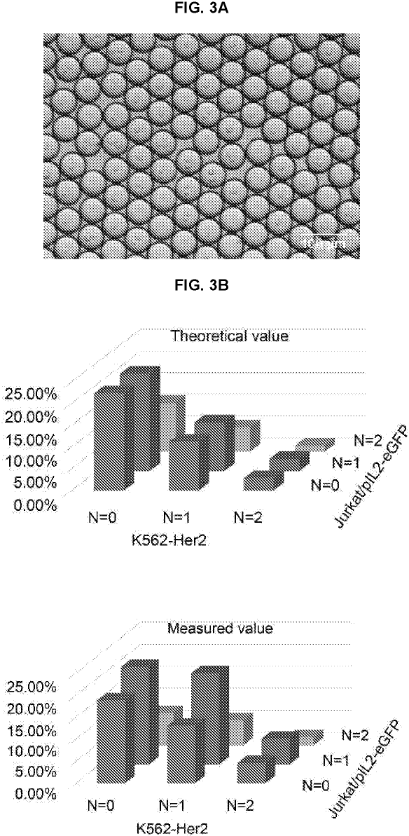

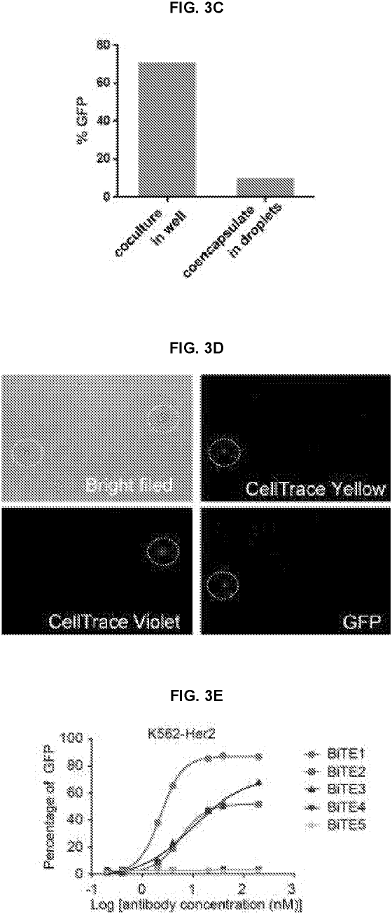

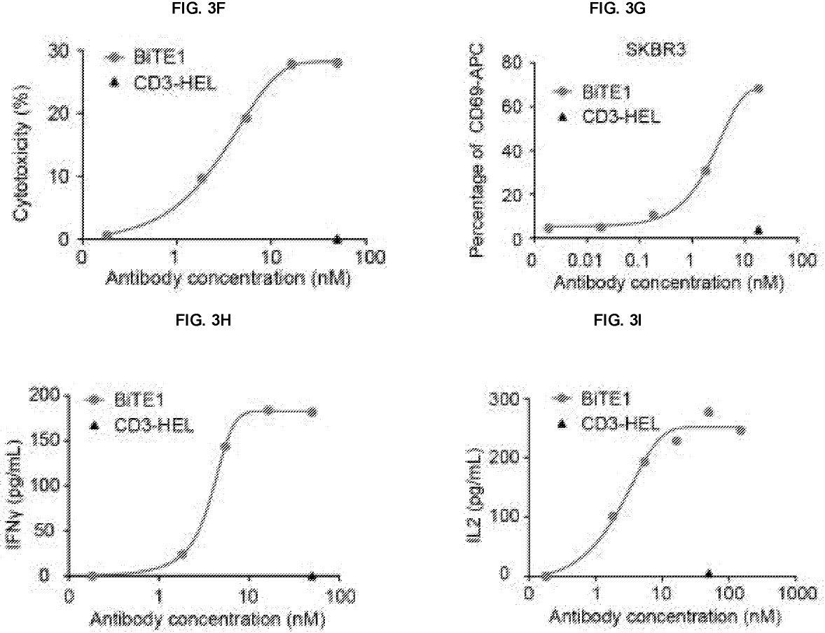

First, the droplets were generated by using microfluidics chip. The droplets were incubated 24 h at 37° C. No merging of droplets were observed, indicating that the droplets were stable (FIG. 3A).

Next, anti-Her2/anti-CD3 BiTE antibody was used as a model for the development and optimization of the droplet-based assay. The BiTE antibodies were created by linking single chain antibodies, one binding to CD3 on T cells and the other to a surface antigen on the target cell (25).

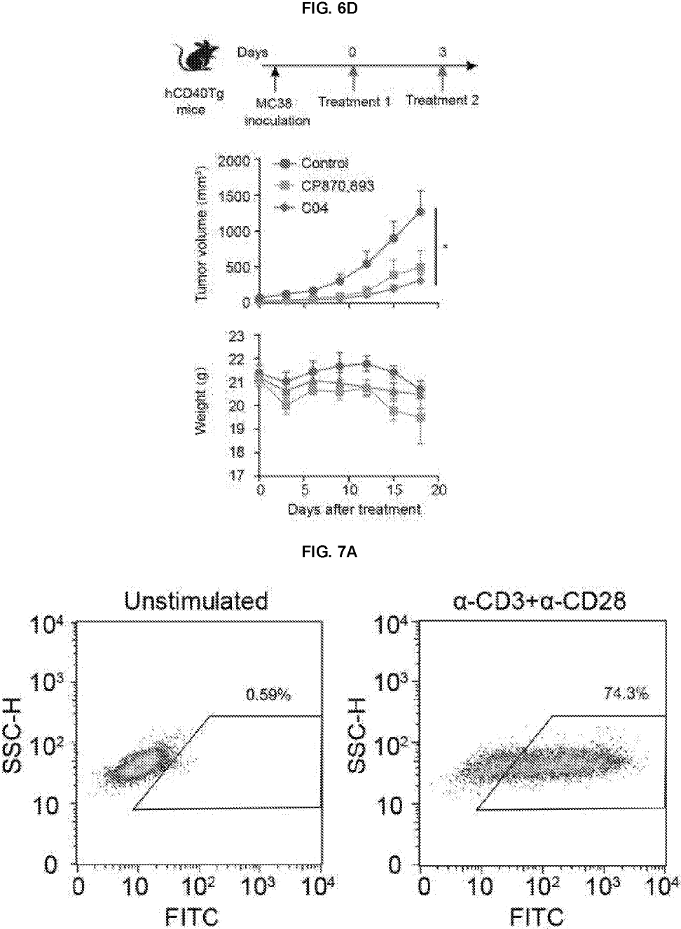

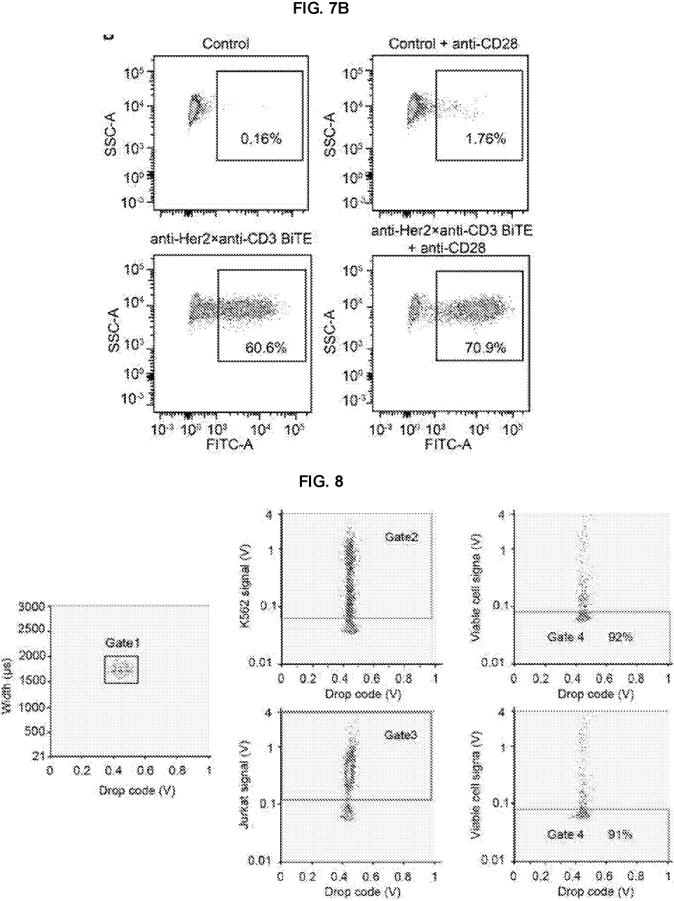

Specifically, trastuzumab-derived anti-Her2 scFv were fused to blinatumomab-derived anti-CD3 scFv to generate an anti-Her2/anti-CD3 positive control. Her2-overexpressing K562-Her2 cells were then infected with the anti-Her2/anti-CD3 positive control lentivirus at a low MOI (<0.1) so that less than 10% of the cells were infected. The K562-Her2 cells played a dual role to express the BiTE antibody and to provide Her2-mediated crosslinking of the secreted antibodies. Individual infected K562-Her2 cells were co-encapsulated with a Jurkat / pIL2-eGFP reporter cell (FIGS. 7A-7B) with λ of 0.5 antibody secreting cell and 1 reporter cell per droplet. The number of cells per droplet was analyzed based on the image, and the results are in good agreement with double Poisson distribution (FIG. 3B).

After 16 hrs of incubation, the droplets were re-injected to the sort chip to analyze the activation of reporter cell in the droplet. About 9.5% of the reporter cells were activated, suggesting high efficiency of reporter cell activation by the secreted antibody in the droplet. This may be due to the extreme high antibody concentration in the very small volume of each droplet. In contrast, when the anti-Her2/anti-CD3 lentivirus infected K562-Her2 cells were cocultured with the reporter cells in the plate well, 72.7% of the reporter cells were activated after 16 hrs of incubation (FIG. 3C).

The difference between the two conditions demonstrated that antibody secreted by cells in the droplet can not transfer between different droplets, thus the system can be used for library screening. In addition, NucGreen Dead 488 were used to track the cell viability in droplets. Both K562 and Jurkat cells in droplets have high viability around 90% after 16 hrs of incubation (FIG. 8).

### Example 2 Functional Screening of Anti-Her2 / Anti-CD3 Bispecific Antibody

The method of the invention was used here to conduct a function-based screening for anti-Her2/anti-CD3 bispecific antibodies.

Because phage display technology can interrogate a much larger diversity space than is possible in eukaryotic systems, Her2-binding antibodies were first selected from an antibody phage display library. A naïve human single chain Fv (scFv) library with size of 10¹⁰ members was panned against the Her2 protein. One rounds of panning were performed, and the scFv genes in phagemid were subcloned into a lentiviral vector, which contained fixed blinatumomab derived anti-CD3 scFv gene to express anti-Her2/anti-CD3 BiTE antibody. The size of the combinatorial bispecific antibody library is about 10⁵ members.

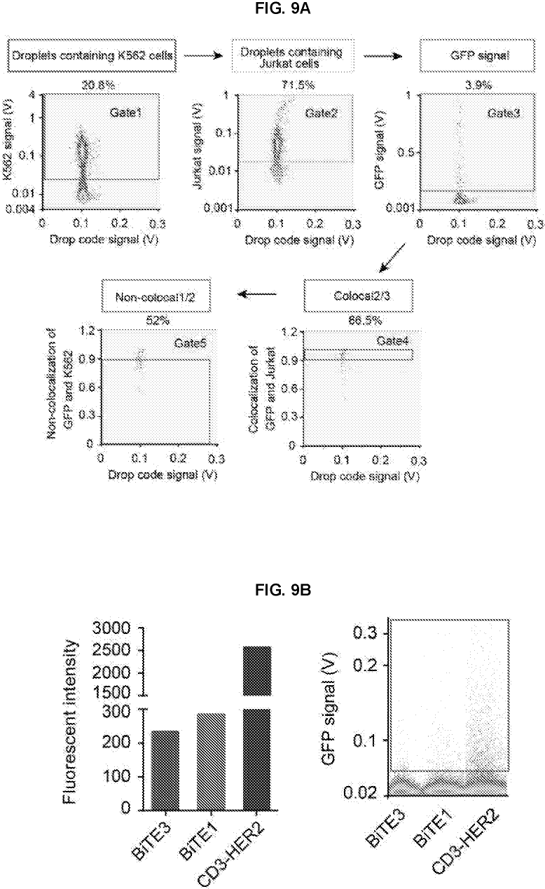

K562-Her2 cells were infected with the BiTE antibody lentivirus library at low MOI to ensure most cells were infected by only one virus and produced a single type of bispecific antibody. Prior to cell encapsulation, K562-Her2 cells were stained with CellTrace Violet, and Jurkat/pIL2-eGFP cells were stained with CellTrace Yellow. Each individually infected K562-Her2 cell was co-encapsulated with a Jurkat/pIL2-eGFP reporter cell into the same droplet. After 16 hrs of incubation, the droplets were sorted based the following gating strategy (FIG. 9A): first, select droplets for the presence of K562-Her2 and Jurkat/pIL2-eGFP cells based on the cell staining fluorescence signals; then select droplets if the reporter cell inside was activated based on GFP signal; and finally, select the droplets if the signal of GFP was colocalized with the reporter Jurkat cell staining signal.

Eleven millions of droplets were imaged and about 0.26% of the droplets were sorted, and a representative image of the sorted droplet were shown (FIG. 3D). The anti-Her2 scFv genes were amplified from the sorted cells and cloned into mammalian expression plasmid. Twenty clones were picked for Sanger sequencing, and five bispecific antibodies appeared more than once. The purified bispecific antibodies were added into the Jurkat/pIL2-eGFP reporter cell in coculture with K562-Her2. Three out of five antibodies (BiTE-1, BiTE-2, BiTE-3) can activate the reporter cell in the presence of K562-Her2 (FIG. 3E).

To assess the antitumor activity of BiTE1, we performed in vitro cytotoxicity assays by coculturing PBMCs and HER2-expressing SK-BR-3 breast cancer cells. The results showed that lysis of SK-BR-3 cells only occurred when they were incubated with BiTE1, not with the control antibody anti-CD3×anti-HEL (hen egg lysozyme, HEL) (FIG. 3F). Flow cytometry analysis of T cells in the coculture system showed that BiTE1 stimulated expression of the activation marker CD69 on T cells (FIG. 3G). Moreover, BiTE1 induced dose-dependent increases in IFN-γ and IL-2 in the supernatants (FIGS. 3H and 3I).

To validate the potential of the method to discriminate between strong and weak hits, trastuzumab-derived anti-Her2×anti-CD3 positive control, BiTE1 and BiTE3 in descending order of potency were each cloned into lentiviruses. Cells infected with each of these bispecific antibodies encoding lentivirus were coencapsulated with the reporter cell, and droplets were analyzed for the activation of the reporter cell. The order of droplet intensity was as follows: trastuzumab-derived anti-Her2×anti-CD3 positive control > BiTE1≈BiTE3 (FIG. 9B). This work demonstrates the capability of the technology to discriminate between weak and strong hits, which enables fast enrichment of high potency hits using a more stringent gating strategy.

Overall, the results demonstrate that this technology allows combinatorial screening and profiling of large numbers of bispecific antibodies.

### Example 3 Screening of CD40 Agonist From a Spike-In Library

This experiment further validates the utility of this function-based screening method, by identifying CD40 agonistic antibodies.

CD40 is a promising drug target for cancer immune therapy (5). Activation of CD40 on antigen presenting cells (APCs) results in improved antigen processing and presentation, and cytokine release, which enhances T cell response.

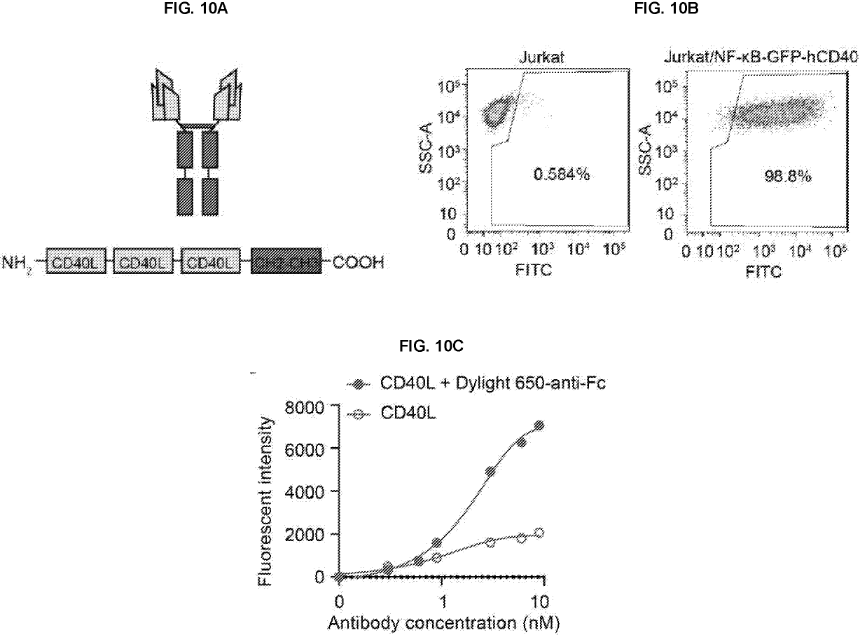

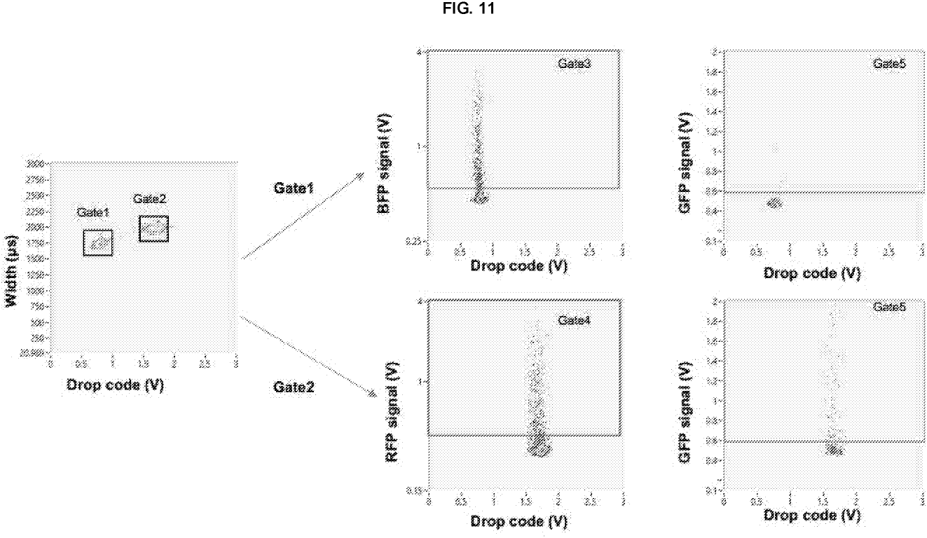

Human Jurkat T cells were engineered to constitutively express human CD40 and express GFP controlled by NF-κB response elements. The reporter cell line express GFP when the CD40 is activated (FIG. 10B).

HEK293T cells co-expressing red fluorescence protein (RFP) and hexameric form of CD40L was used as positive control (CD40L cell). HEK293T cells co-expressing blue fluorescence protein (BFP) and a non-related hen egg lysozyme (HEL) antibody was used as negative control (HEL cell).

The CD40L or HEL cells were co-encapsulated with the Jurkat / NF-κB-GFP-hCD40 reporter cells into droplets. After 16 hrs of incubation, 24% of the CD40L cell-containing droplets exhibited GFP fluorescence signal while the HEL cell and reporter cell co-encapsulating droplets showed clean background of activation of the reporter cell (0.5%) (FIG. 11).

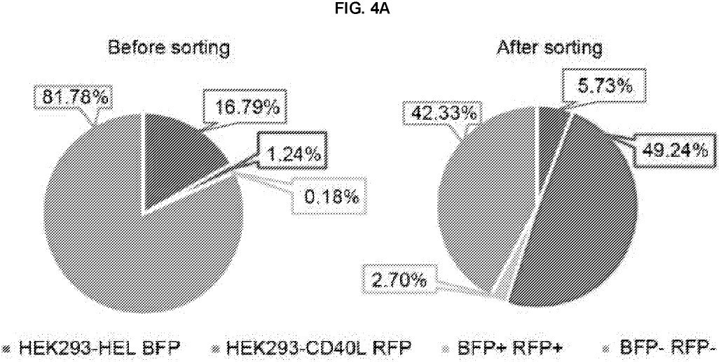

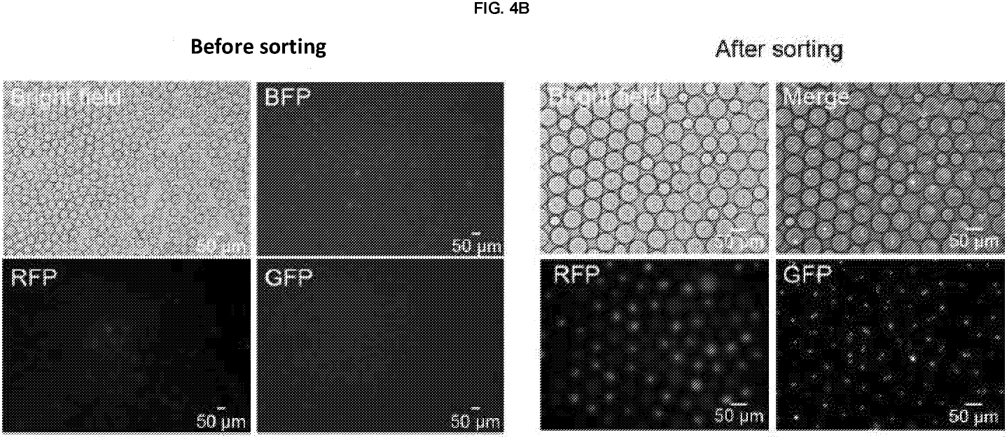

CD40L cells were spiked into a 10-fold excess of HEL cells. The mixture of CD40L cells and HEL cells were co-encapsulated with the Jurkat / NF-κB-GFP-hCD40 reporter cells with a mean λ of 0.5 protein secreting cell per droplet. The droplets which contained activated reporter cell were sorted based on green fluorescence. Before sorting, only 0.94% of the mixed cell population were RFP positive, 16.58% of cell were BFP positive, and the rest droplets were empty or contained only reporter cell, whereas after the sort the percentage of droplets containing RFP positive CD40L cell increased to 52.57%, where 49.48% RFP positive and 3.09% RFP and BFP double positive (FIG. 4A). Most droplets containing both RFP positive CD40L cells and activated GFP reporter cell after sorting (FIG. 4B).

### Example 4 Screening of CD40 Agonist Antibody Using the Function-Based Screening Method

CD40 binding antibodies were first selected from an antibody library using phage display technology. The scFv genes in phagemid were subcloned into lentiviral vector that contained IgG1 Fc gene to express scFv Fc fusion protein. The library size was about 10⁵. HEK293T cells were infected with lentivirus library at low MOI to ensure most cells were infected by only one virus and produced one type of monoclonal antibody.

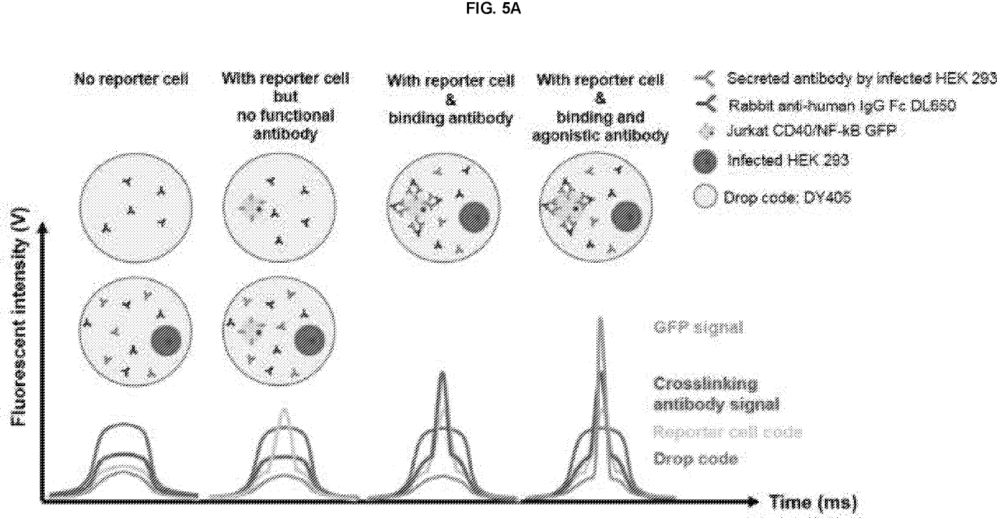

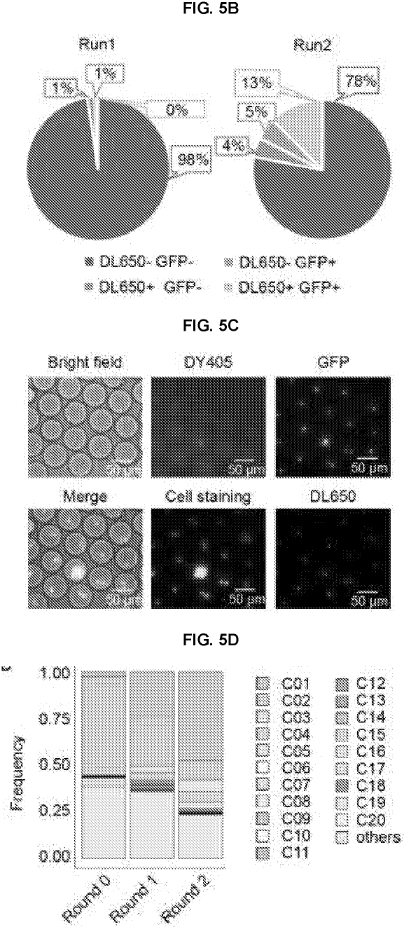

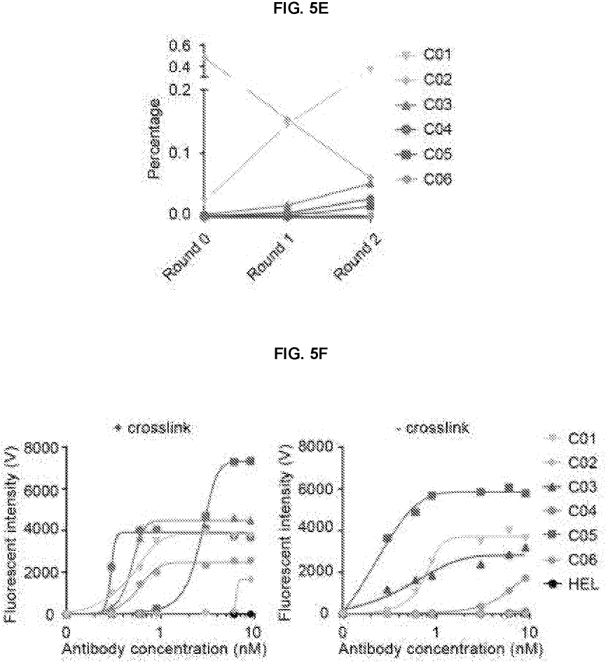

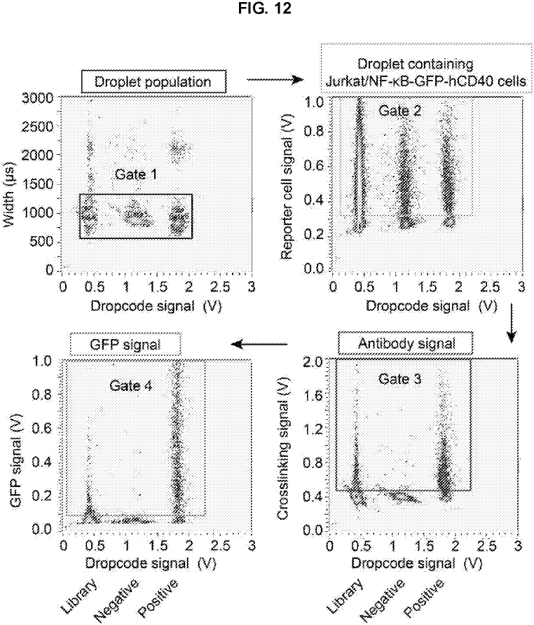

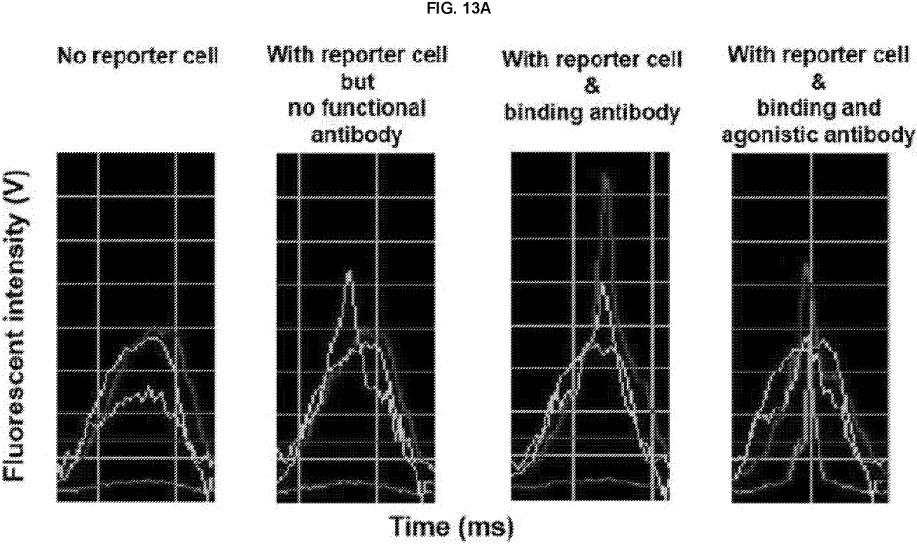

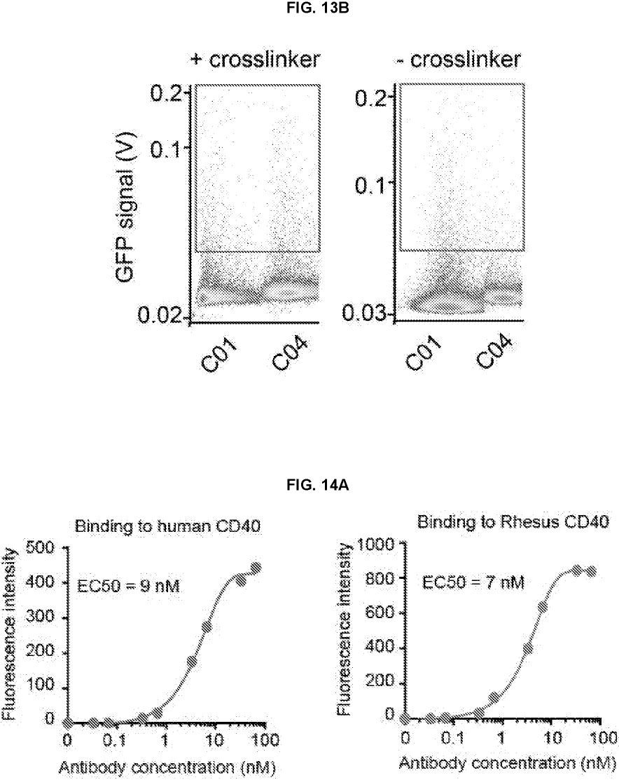

To screen CD40 agonist antibodies, the antibody-producing cells were co-encapsulated with CellTrace Yellow prestained Jurkat / NF-κB-GFP-hCD40 reporter cell, and Dylight647-conjugated secondary antibody in droplets. The reporter cells were also co-encapsulated with soluble hexameric CD40L protein and anti-HEL antibody as positive and negative controls. Droplets of different populations (positive control, negative control, and the screening population) were coded by different concentrations of DY405. After 16 hrs of incubation, the droplets were sorted based on the following criteria (FIG. 12). The droplets of the screening population were first gated based on the intensity of DY405. CellTrace Yellow fluorescence signal showed the presence of reporter cell in the droplet. The Dylight647 fluorescence signal indicated binding of the secreting antibodies to CD40 on the surface of the reporter cell, and GFP fluorescence signal peak indicated activation of the reporter cell (FIG. 12). The droplets displayed different patterns based on their fluorescence signals (FIG. 5A, and FIG. 13A). 13 million and 4.8 million droplets were imaged for the 1st round and the 2nd round of screening, respectively. Whereas only 0.29% droplets were Dylight647 and green fluorescence double positive for the 1^{st} round of screening (out of the droplet containing Jurkat cell signal), this value increased to 12.5% for the 2^{nd} round of screening (FIG. 5B). The droplets before and after sorting were imaged and the number of Dylight647 and green fluorescence double positive droplets dramatically increased after the sorting (FIG. 5C).

The scFv genes were amplified from the cells after each round of sorting and were subject to the third-generation sequencing. Circular consensus sequences from these reads were generated, filtered by quality. Full-length scFvs were identified in 41,971 reads. Considering the errors introduced by PCR or sequencing, the similar (at 95% similarity) scFv sequences were grouped into 602 scFv clusters and consensus scFv sequences of each cluster were created. Comparison of the sequence frequency in each round revealed that some scFvs were enriched while some scFvs were eliminated during the selection (FIG. 5D). The top 6 scFv clusters were chosen after the 2^{nd} round of selection based on enrichment factor in each rounds. The frequency of scFv clusters C01, C03, C04, C05, and C06 were low before sorting, and showed roundwise increase during the selection process. In contrast, scFv cluster C02 were highly abundant before sorting, but its frequency was significantly reduced after sorting (FIG. 5E).

The scFv sequences of C01, C03, C04, C05, and C06 are listed below, with the heavy chain CDR sequences double underlined and in bold font, and the light chain CDR sequences double underlined and in italic font.

C01:

MAQVQLVESGGGLVQPGRSLRISCAGSGFTFGDSA MHWVRQAPGKGLEWV
SGISRNSDTI VYADSVKGRFTISRDNAKNSLYLQMNSLRAEDTALYYCAR RSGDHHAMDV WGPGTTVTVSSGGGGGGSETTLTQSPATLSLSPGERATLS
CRASQSVNTY LAWYQQKPGQAPRLLMYDSS SRATGIPDRFSGSGSGTDFT
LTISRLEPEDFAVYYCQQYSTVPLT FGGGTKVDIKR

C03:

MAQVQLVESGAEVKKPGASVKVSCKASGYTFTGYY MHWVRQAPGQGLEWM
GWISAYNGNT NYAQKLQGRVTLTRDTSTSTVYMELSSLRSEDTAVYYCAR AKKIRGYSYGGFDY WGQGTTVTVSSGGGGGGSQSALTQPASASGSPGQSV
TISCTGTSSDVGGYNY VSWYQQHPGKAPKLLIYEVN KRPSGVPDRFSGSK
SDNTASLTVSGLQAEDEADYYCSSYAGSDNSYV FGTGTKLTVLG

C04:

MAQVQLVQSGAEVKKPGASVKVSCKASGYTFTGYY MHWVRQAPGQGLEWM
GWINPNSGGT NYAQKFQGRVTMTRDTSISTAYMELSRLRSDDTAVYYCAR ERVGATPTYYYYMDV WGKGTTVTVSSGGGGSGGGGSGGGGSDVVMTQSPF
SLPVTPGEPASISCRSSQSLLHSNGHNY LDWYVQKPGQSPQLLIHLGS NR
ASGVPDRFSGGGSGTDFTLKISRVEAEDVGVYYCMQALQTPFT FGPGTKV
DIKR

C05:

MAQVQLQQSGPGLVKPSQTLSLTCAISGDSVSSNTAA WNWIRQSPSRGLE
WLGRTYYRSKWYN DYAVSVKSRITINPDTSKNQFSLQLNSVTPEDTAVYY
CARQVQLERRHAFDI WGQGTLVTVSSGGGGGSQSALTQPASVSGSPGQSI
TISCTGTSSDVGGYNY VSWYQQHPGKAPKLMIYEVS NRPSGVSNRFSGSK
SGNTASLTISGLQAEDEADYFCSSYTSSSTVVI FGGGTKVTVLG

C06:

MAQVQLLQSGGGVVQPGRSLRLSCAASGFTFSSYG MHWVRQAPGKGLEWV
AVISYDGSNK YYADSVKGRFTISRDNSKNTLYLQMNSLRAEDTAVYYCAK VIRGSSGWSDAFDI WGQGTMVTVSSGGGGSGGGGSGGGGSQAVLTQPSSA
SGTPGQRVTISCSGSSSNIGSHT VSWYQQLPGTAPKLLIYSTD QRPSGVP
DRLSGSKSGTSASLTISGLQSEDEAHYYCAAWDDSQNKSLV FGGGTQLTV
LG

TABLE1

EC50 of selected CD40 agonist antibodies.

| CD40 agonist | C01 | C02 | C03 | C04 | C05 | C06 |
|---|---|---|---|---|---|---|
| EC50 without crosslinker (nM) | 7 | - | 6 | - | 3 | - |
| EC50 with crosslinker (nM) | 6 | - | 5 | 3 | 30 | 5 |

Based on the HCVR and LCVR CDR sequences, the genes of full length IgG C01, C02, C03, C04, C05, and C06 were synthesized and recombinant antibodies were expressed and purified. The activity of the antibodies was tested using the Jurkat / NF-κB-GFP-hCD40 reporter cell. The reporter cells were stimulated with different concentrations of antibody in the presence or absence of the crosslinking secondary antibody. Antibodies C01, C03, C04, C05, and C06 activated the reporter cell line. The activity of C01, C03, and C05 was independent of crosslinking, while C04 and C06 activated the reporter cell in a crosslinking dependent manner (FIG. 5F). Signal intensities between Fc-dependent C04 and Fc-independent C01 in the droplet-based assay were compared, and the results supported that the differential Fc-dependency of these hits (FIG. 13B). The Fc receptor independent activity of CD40 agonist antibody is of concern because this feature underlies possible systemic adverse events. Therefore, the potent Fc receptor-dependent antibody C04 was chosen for further characterization.

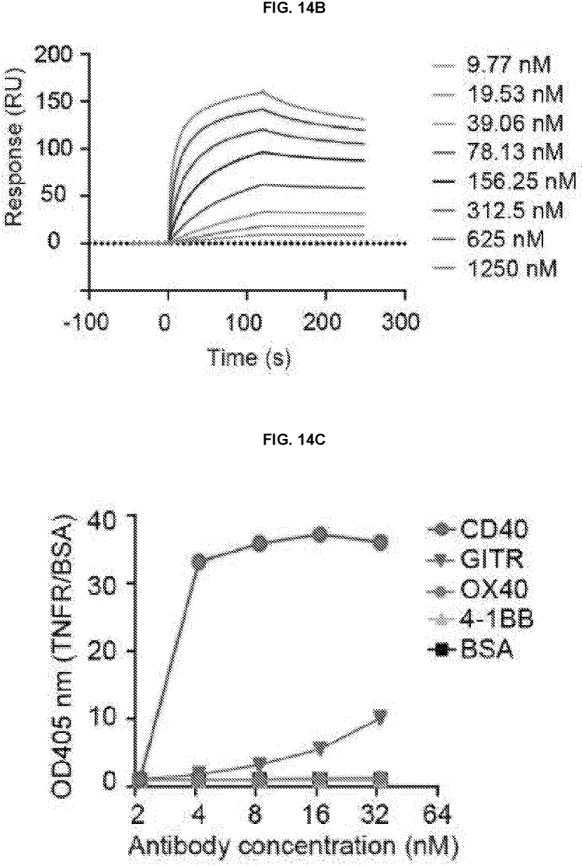

Flow cytometry results showed that antibody C04 bound to human, rhesus macaque and cynomolgus monkey CD40 with similar affinity (FIGS. 14A and 14B). In addition, ELISA results confirmed that antibody C04 specifically bound to CD40 rather than other TNF receptor superfamily members such as GITR, OX40 and 4-1BB (FIG. 14C).

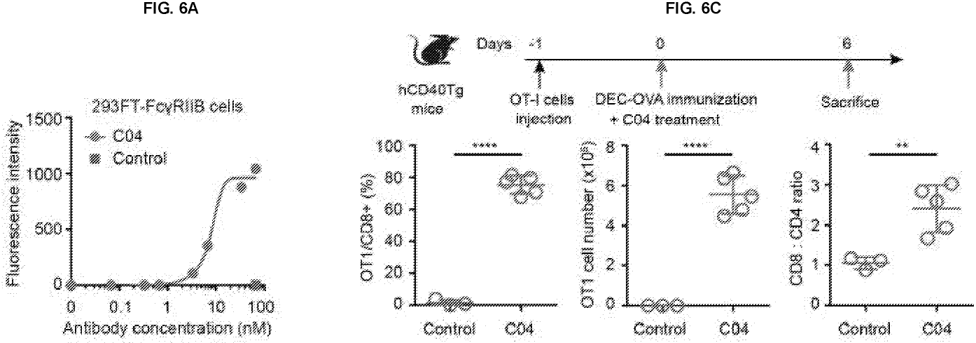

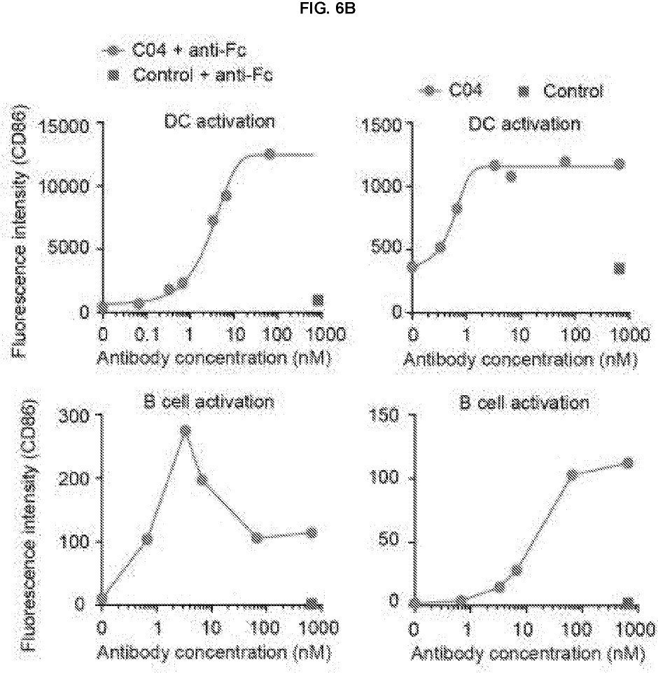

Since FcγRIIB expressed on tumor infiltrating myeloid cells is required for agonistic activity of the crosslinking-dependent CD40 agonist antibodies, the FcγRIIB dependency of antibody C04 was assessed. The Jurkat / NF-κB-GFP-hCD40 reporter can be stimulated by C04 in the presence of FcγRIIB-overexpressing cells. The results revealed that the agonism of C04 was FcγRIIb dependent (FIG. 6A).

To investigate whether C04 can promote the activation of CD40-positive APCs, human dendritic cells (DCs) and B cells isolated from PBMC were stimulated by C04 in the presence or absence of the crosslinking antibody. Flow cytometry analysis showed that C04 stimulated the expression of activation marker CD86 on DCs and B cells, and anti-Fc mediated crosslinking could further enhance the agonistic activity (FIG. 6B).

The immunostimulatory activity of antibody C04 was further tested in mouse humanized for CD40 and FcγRs. The recipient CD40/FcγR humanized mice were adoptively transferred with ovalbumin (OVA)-specific OT-I cells, and OVA were delivered to dendritic cells by i.p. injection of the chimeric anti-DEC205 antibody conjugated to OVA (DEC-OVA) together with either C04 or anti-HEL antibody. Mice treated with C04 displayed significantly increased percentage of OT-I cells among CD8⁺ T cells compared to the mice treated with control antibody, demonstrating C04 had robust adjuvant effect (FIG. 6C).

In addition, antitumor efficacy of C04 was assessed in syngeneic tumor models. When MC38 tumors were established (~100 mm³), mice were treated with C04, CP-870,893(Pfizer) or anti-HEL antibodies. C04 displayed comparable anti-tumor activity as CP-870,893, which is the most potent CD40 agonistic antibody among those tested in clinical trials to date. In addition, treatment with C04 didn’t cause severe reduction of body weight as CP-870,893 did, suggesting that C04 had a favorable toxicity profile (FIG. 6D).

### Example 5 Discussions

Here, a droplet-based microfluidics platform for the functional screening of millions of antibodies was described. The platform shares some key features with the most efficient selection methods, such as phage display. First, the genotype and phenotype linkage was maintained throughout the whole process. Second, the product from one round can be directly amplified and used as the input in the next round of selection. Thus, multiple rounds of iteration allow the enrichment of rare hits. Compared to the conventional method of individually expressing and assaying thousands of antibodies, the throughput of this platform increased that limit to 10 million. This is especially useful for the development of next-generation cancer immunotherapies, such as agonist antibodies or bispecific antibodies, when simple binding assays may be inadequate.

To demonstrate the usefulness of this platform, it was first applied to discover bispecific antibodies and agonist antibodies, whose development was limited by low diversity and/or low throughput and potentially biased screening.

Bispecific T or NK cell engagers (BiTEs or BiKEs) hold great promise for cancer treatment, and a growing number of BiTEs and BiKEs are making their way through various stages of development. To identify the optimal BiTE or BiKE, a bispecific antibody library was constructed to address the complexity of the array of tumor antigen targeting antibodies. The large number of bispecific antibodies in a given library can exceed the throughput of existing methods. The described approach provides significant opportunities to screen unprecedented numbers of molecules of different formats and compositions, such as antibodies and nonantibody protein scaffolds. However, the method by itself cannot guarantee the generation of functional hits of high potency, and the successful isolation of potent antibodies also depends on the existence of such hits in the library. Similar to other in vitro display methods, the efficiency of drug discovery will scale with library size.

A single round of screening was performed to identify anti-Her2×anti-CD3 antibodies. Such a single round could be potentially sufficient for some targets, but the false positive rate could be high in certain screenings. A few reasons could contribute to the false positive rate. First, the local concentration of antibody in the droplets is very high, and may result in an increased tendency to form high-molecular-weight aggregates and nonspecific activation of the reporter cell. Second, the gating strategy may not be stringent enough to exclude all-false positive events. As used in well-established methods, such as phage display, iterative rounds of screening may help to enrich the true hits and eliminate false positive hits. In addition, more stringent gating strategies can be applied in screening.

Research into costimulatory receptor agonists has been reignited over the last decade due to substantial advances in the field of immunoncology. Costimulatory receptors are expressed on a number of immune cell types, including T cells, B cells and natural killer (NK) cells, as well as APCs, and engagement of these receptors promotes immune cell function, proliferation and survival. Nevertheless, there are no general rules to guide the screening of agonist antibodies. For example, a panel of antibodies binds to the same or similar epitopes of the Fas receptor but results in different biological effects, with some acting as agonists and others as antagonists . The intrinsic complexity of agonist antibodies requires screening as many antibodies as possible. The instant unique platform of the disclosure was used to screen for CD40 agonist antibody and discovered a few potent CD40 agonist antibodies, most of which were too rare (<0.02% frequency) to be discovered using a conventional screening platform.

The instant method of the disclosure can also be applied to high-throughput analysis of cell-cell interactions. For example, this method is applicable where DC cells infected with a lentivirus library encoding neoantigens are coencapsulated with tumor infiltrating T cells to map the pairs of cognate antigens and T cell receptors (TCRs). This method can also be adapted to screen different types of molecules, such as cytokines. In addition to application in drug discovery, identification of intercellular signaling pathways has become an actively growing field. Combining our technology with the power of CRISPR/Cas9 library screening could enable the deciphering of cell-cell communications at scale. The innovative applications of this activity-based selection method have been limited only by the imagination of the users.

In summary, the instant disclosure describes a unique high-throughput platform for function-based screening of up to millions of antibodies. With the capability to screen millions of antibody-producing cells without any presumptions other than the key function used for screening, this may revolutionize next-generation cancer immunotherapy drug discovery and development, as well as advance basic research involving cell-to-cell interactions.

Materials and certain experimental details for the Examples above are provided herewith.

### Cell Culture

HEK293FT cells (Thermo Fisher Scientific, R70007) were cultured in DMEM (Thermo Fisher Scientific). Jurkat cells (ATCC, TIB-152) were cultured in RPMI 1640 medium (Thermo Fisher Scientific). SK-BR-3 cells (ATCC, HTB-30) were cultured in McCoy’s 5A (modified) medium (Biological Industries). All the culture medium was supplemented with 10% fetal bovine serum (Biological Industries), 1 × nonessential amino acids, 100 U/ml penicillin, 100 µg/ml streptomycin and 12.5 mM HEPES (Thermo Fisher Scientific). HEK293F cells (Thermo Fisher Scientific, R79007) were suspended and cultured in FreeStyle™ 293 Expression Medium (Thermo Fisher Scientific). All cells were maintained in a CO2 incubator at 37° C.

### IL2 Reporter Cell Line

Jurkat / pIL2-eGFP was developed to monitor activation of T cells. The immortalized T cell leukemia Jurkat cell line was transfected with vector carrying eGFP reporter gene under the control of full-length IL-2 promoter (from -648 to -1 upstream of IL-2 translation initiation codon). After co-stimulation by anti-CD3 and anti-CD28 antibodies, cells expressing high level of GFP were sorted into wells of a 96-well plate using FACS, and individual clones of Jurkat / pIL2-eGFP cell line were characterized.

### CD40 Reporter Cell Line

Jurkat / NF-κB-GFP-hCD40 reporter cell was developed to monitor activation of CD40. The Jurkat cell was transfected with a vector carrying an eGFP reporter gene under the transcriptional control of NF-κB response element. After stimulation by 10 ng/mL TNFα (Sino Biological), cells expressing high level of eGFP were sorted by FACS, and the Jurkat / NF-κB-eGFP cell line were characterized.

The resulting Jurkat / NF-κB-eGFP cell line was then infected with lentivirus expressing full-length human CD40. After stimulation by 100 nM hexameric CD40L Fc fusion protein, individual cells with high level of GFP signal were sorted into the wells of a 96-well plate by FACS, and individual clones of the Jurkat / NF-κB-GFP-hCD40 cell were characterized.

### Microfluidic Chip Fabrication

All microfluidic chips were fabricated in polydimethylsiloxane (PDMS) polymer (Sylgard 184 elastomer kit; Dow Corning Corp) using the standard soft lithography as described (31). Masters were made using one layer of SU-8 photoresist (MicroChem). The depth of the two devices is 40 ± 1 µm to allow the droplet generating or flowing in a monolayer format. For device ii, the PDMS is bonded to a piezoelectric substrate (Y¹²⁸-cut Lithium niobate wafer) where an golden interdigital electrode is patterned with standard lift-off technology and aligned with the fluidic channel above. Microfluidics devices were treated before use with 1% v/v 1^{H}, 1^{H}, 2^{H}, 2^{H}-perfluorodecyltrichlorosilane (Alfa Aesar) in Novec HFE7500 fluorinated oil (3 M) to prevent droplets from wetting the channel walls.

### Droplet Production, Collection, and Incubation

Aqueous phases containing infected cells and reporter cells were co-flowed and partitioned into droplets with hydrodynamic flow focusing in dripping mode on a microfluidic chip (FIG. 2A). The nozzle is 15 µm wide, 40 µm deep and 10 µm long. The continuous phase was Novec HFE-7500 fluorinated oil (3 M) containing 2% w/w 008-FluoroSurfactant (RAN Biotechnologies). Pressure pumps (Fluigent) were used to generate monodispersed droplets of 100 pL±10 pL at 5000 Hz. The droplets were collected into a 10 mL tube and incubated at 37° C. in 5% CO2 to allow antibody secretion and subsequent activity to occur within each droplet prior to screening.

### Microfluidic Droplet Screening and Recovery

Droplet fluorescence analysis and sorting operations were performed on a dedicated droplet microfluidic station, similar to that described by Mazutis et al. (15). Pressure pumps (Fluigent) were used to inject the collected droplets into the sorter device (FIG. 2C) at a frequency of 1000-3000 Hz. The sorter device was mounted on an inverted microscope (ASI Microscope) equipped with a 940 nm LED illumination source (Thorlabs M940L3) and a fixed focus laser line (solid-state laser of wavelength 405 nm, 488 nm, 561 nm or 635 nm, Omicron) with photomultiplier tube bandpass filters of 440/40-25 nm, 525/40-25 nm, 593/46-25 nm and 708/75-25 nm (Hamamatsu).

The fluorescence of each droplet was measured as the droplet flowed past an observation constraint in the microfluidic channel where the laser line was positioned. The emitted fluorescence was detected with PMTs, converted into corresponding signal output voltages, and recorded by the data acquisition card (FPGA PCIe-7842R). These voltages were then processed by the card and custom Lab View software to identify droplets according to their fluorescence intensity and size. These characteristics were used to determine whether each droplet should be sorted.

Droplets were sorted based on surface-acoustic wave deflection as described by Frank et al. with a GHz-signal generator (Wavetek, Model 3010) (21, 42). Sorted droplets were collected in a 1.5 mL Eppendorf tube. Cells were recovered by adding 100 µL DMEM culture medium, followed by 100 µL of 1H, 1H, 2H, 2H-perfluoro-1-octanol (Sigma, 37053), and then cells were pooled and centrifuged at 400 g for 5 min at 4° C. for subsequent steps, such as subculture or DNA sequencing.

### Phage Display

A human naïve scFv library was constructed from the PBMC of 30 healthy donors with standard protocols. The phage library was incubated with biotinylated CD40-Fc fusion protein or Her2 recombinant protein (Acro biosystems) for 2 hours at room temperature (RT), and the phage-antigen complex was captured by Dynabeads M280 (Life technologies). The bound phages were eluted by Glycin-HCl (pH 2.2) for 10 min. at RT, and neutralized with Tris-HCl (pH 8.0) to adjust pH to 7.5. The phagemid DNA was isolated using plasmid miniprep kit (Qiagen).

### Lentivirus Library Construction

Both phagemids and lentiviral vector pLV-ef1α-ScFv-Fc were digested with enzyme SfiI. The lentiviral vector and scFv genes were isolated after electrophoresis. ScFv genes were then ligated into the lentiviral vector. The product of ligation reaction were transformed into XL1-Blue competent cells by electroporation, and most of the transformed bacteria were plated on LB Agar plates. The remaining bacteria were serially diluted and plated to estimate the size of the library. Lentiviral plasmid was prepared using plasmid midiprep kit (Qiagen) for lentivirus preparation.

### Lentivirus Preparation

When confluency reached 80%, HEK293T cells were transfected with lentiviral backbone plasmid and packaging plasmids using PEI transfection reagent. The medium was then changed to fresh complete culture medium 6 hrs post transfection. Supernatant containing lentivirus was harvested after 48 hours, centrifuged at 300 g for 5 min. at 4° C., and filtered by 0.45 µm filter to remove cell debris. Viral titer was measured using P24 ELISA kit (Clontech). The virus was aliquoted and stored at -80° C.

### Function Based Screening of Anti-Her2/anti-CD3 Bispecific Antibody Using Microfluidics

Aqueous phase I: Jurkat / NF-κB-eGFP reporter cells were washed with PBS and stained with 1 µM CellTrace Yellow dye for 10 min. at 37° C. The stained Jurkat / NF-κB-eGFP reporter cells were washed twice with RPMI 1640, and then resuspended in cell culture medium (RPMI 1640, 5% FBS, 25 mM HEPES, and 0.1% Pluronic F-68) containing 1 µg/mL anti-CD28 antibody (Invitrogen).

Aqueous phase II: The stable Her2 expressing K562 cells were infected with the lentiviral antibody library. The resulting antibody-secreting K562 cells were washed with PBS, and stained with 1 µM CellTrace Violet dye for 10 min. at 37° C. The stained cells were washed twice with RPMI 1640, and resuspended with cell culture medium containing 200 nM DY647. K562 cells infected with positive control lentivirus were resuspended with culture medium containing 1.5 µM DY647.

Aqueous phases I and II were injected into the droplet generation chip from different inlets and used as the disperse phase. Novec HFE7500 fluorinated oil (3 M) containing 2% w/w fluoro-surfactant (RAN Biotechnologies) was used as the continue phase to produce droplets with average size of 100 pL. The flow rates of aqueous phase I, aqueous phase II, and oil phase were adjusted, so on average one reporter cell and 0.5 antibody secreting cell were co-encapsulated per droplet. During droplet production, the cell suspension was cooled using ice-water to inhibit antibody secretion. The droplets were collected and incubated at 37° C. for 16 hrs.

The droplets were first gated to eliminate coalesced droplets and retain only droplets of desired size. Positive control and the screening population droplets were distinguished based on the different intensity of the fluorescence of DY647. For the screening population, the droplets were selected for the presence of Jurkat reporter cells based on CellTrace Yellow signal, and K562 cells based on CellTrace Violet signal. The FADS was performed to sort the droplets containing Jurkat emitting GFP fluorescence. Finally, droplets with GFP signal colocalized with Jurkat staining signal, but not with K562 signal, were gated and sorted.

The cells were recovered from the sorted droplets by adding 200 µL RPMI 1640 medium supplemented with 10% Fetal Bovine Serum (FBS) and 24% Nycodenz, followed by adding 50 µL 1^{H}, 1^{H}, 2^{H}2^{H}-Perfluoro-1-octanol (Sigma). After mixing the droplets thoroughly and centrifuging them at 300 g for 5 min. at 4° C., the aqueous layer was completely separated and washed with RPMI 1640 medium. The recovered cells were lysed, and their antibody genes were amplified from the cells.

### Anti-Her2/Anti-CD3 Bispecific Antibody in Vitro experiment/Jurkat / IL-2-GFP Reporter Cell Assay

For detecting the activity of anti-Her2 / anti-CD3 BiTE antibody candidates, the Jurkat / IL-2-GFP reporter cells were stimulated with different concentrations of anti-Her2 / anti-CD3 antibodies and 1 µg/mL anti-CD28 antibody in the presence of K562 cells or K562-Her2 cells. After 16 hrs of incubation, GFP expression in the reporter cells was detected by flow cytometry.

### Anti-Her2 / Anti-CD3 Bispecific Antibody Ex Vivo Assay

Primary T cells were isolated from PBMCs using CD3 MicroBeads (Miltenyi, 130-050-101). 1×10⁵ T cells were cocultured with target cells (SKBR3 cells, MDA-MB-231 cells, or HEK293 cells) at a 1:1 ratio. Different concentrations of anti-Her2 / anti-CD3 antibody or control antibody, together with 1 µg/mL anti-CD28 antibody were then added. After 48 hrs of incubation, cells were collected and stained with anti-CD3-FITC (BioLengend, 300406) and anti-CD69-APC (BioLegend, 310910) for 30 min. at 4° C. T cell activation was determined by flow cytometry. Flow cytometry results were analyzed with software Flowjo X.

Cell supernatant was collected to quantify cytokine release and cytotoxicity. IL-2 and INF-γ were measured with ELISA kit according to the manufacturer’s instructions. Cytotoxicity was analyzed by measuring levels of released lactate dehydrogenase (LDH) using the CytoTox 96 non-radioactive cytotoxicity assay protocol (Promega).

### Function-Based Screening of CD40 Agonist Antibody Using Microfluidics

Aqueous phase I: Jurkat-CD40-NFκB reporter cells were washed with PBS and stained with 1 µM CellTrace Yellow dye for 10 min. at 37° C. The stained Jurkat-CD40-NFκB reporter cells were washed twice with DMEM, and then resuspended at 20 million cells/mL with cell culture medium (DMEM, 5% FBS, 25 mM HEPES, and 0.1% Pluronic F-68) containing 16.67 nM Dylight647-conjugated goat anti-human Fc IgG and 24% Nycodenz. The secondary antibody DyLight 650-conjugated goat anti-human Fc IgG was used to mimic the crosslinking action by the Fc receptor.

Aqueous phase II: For the screening population, the HEK293 cells were infected with the lentiviral antibody library and resuspended with cell culture medium containing 500 nM DY405. For positive control droplets, HEK293 cells were resuspended with cell culture medium containing soluble hexameric CD40L protein and 1,500 nM DY405. For negative control droplets, HEK293T cells were resuspended with cell culture medium containing anti-HEL antibody and 2,500 nM DY405.

Aqueous phases I and II were then injected into the droplet generation chip from different inlets and used as the disperse phase. Novec HFE7500 fluorinated oil (3 M) containing 2% w/w fluoro-surfactant (RAN Biotechnologies) was used as the continuous phase to produce droplets with an average size of about 100 pL. The flow rates of aqueous phase I, aqueous phase II, and oil phase were adjusted, such that on average, 1 reporter cell and 0.5 antibody-secreting cell were co-encapsulated per droplet. During droplet production, cell suspension was cooled using ice-water to inhibit antibody secretion. Droplets were collected and incubated at 37° C. for 16 hrs.

The droplets were first gated to eliminate coalesced droplets and retain only droplets of the desired size. Negative control droplets, positive control droplets and screening droplets were distinguished based on their different intensity of the blue fluorescent dye DY405. For screening population, the droplets were selected for the presence of Jurkat reporter cells in the droplet based on the yellow fluorescence of CellTrace Yellow dye. Finally the FADS was performed to sort the droplets containing Jurkat emitting fluorescence of Dylight647 and GFP. The cells were recovered from the sorted droplets by adding 200 µL DMEM medium supplemented with 10% FBS and 24% Nycodenz, followed by adding 50 µL 1^{H}, 1^{H}, 2^{H}, 2^{H}-Perfluoro-1-octanol (370533, Sigma). After mixing thoroughly and centrifugation at 300 g for 5 min at 4° C., the aqueous layer was completely separated and washed with DMEM medium. The cells were then resuspended with DMEM medium containing 10% FBS and 1% PS (Penicillin Streptomycin) and then cultured for 1-2 weeks.

### Bioinformatic Analysis of the PacBio Sequencing Results

Single molecule real-time (SMRT) sequencing platform (Pacific Biosciences) generates long sequencing read with an average read length of ~20 kb, which can sequence an scFv (subreads) more than 10 times. Circular consensus sequencing program from PacBio SMRTportal software (version 4.1.0) takes multiple subreads of the same SMRT bell sequence and combines them, employs a statistical model, and produces one high quality circular consensus sequence (CCS). After CCS from long sequencing reads were generated, scFv flanking sequences were trimmed, scFv DNA sequences were translated into protein using CLC genomics workbench (version 11.0.1), and CDR1-3 regions for heavy and light chains were identified by IgBLAST (version 1.15.0). CD-hit (version 4.8.1) was used to group scFvs at protein similarity 95%. Frequency of scFvs for each round were calculated in java program, respectively. ScFvs that appeared in only one sample were removed because those are likely PCR artifact products. The barplot were draw by R packages ggplot2 (version 3.2.1).

### Protein Expression and Purification of Fill Length IgG

Equal amounts of heavy chain and light chain expression plasmids were co-transfected into 293F cells. Five days after transfection, the transfected cells were centrifuged at 3,000 rpm for 10 min at 4° C., and the supernatants were harvested and passed through a 0.45 µm filter. Antibodies were purified with HiTrap Protein A column (GE) using ÄKTA purifier chromatography system.

### CD40 In Vitro Experiment

### Species Cross Reactivity of Antibody

Cross-reactivity of C03 was assessed by flow cytometry analysis. Briefly, HEK293T cells were transiently transfected with human or rhesus macaque CD40 expressing vector using PEI (polyscience). After 48 hours, HEK293T-hCD40 or HEK293T-rCD40 cells were incubated with different concentrations of antibody at RT for 30 min. Then the cells were stained with AlexFuor488-conjugated goat anti human Fc (Life technologies) at RT for 30 min., and analyzed by flow cytometry. Fluorescence intensity equals to the percentage of GFP positive cells multiplied by Median Fluorescence Intensity(MFI). The fluorescence intensity was plotted against the antibody concentrations using software GraphPad Prism.

### Surface Plasmon Resonance (SPR) Analysis

SPR experiments were performed with a Biacore T200 SPR system (GE Healthcare). In brief, experiments were performed at 20° C. in HBS-P⁺ buffer (0.01 M HEPES, 0.15 M NaCl, and 0.05% v/v Surfactant P20). Anti-his antibody was immobilized on Series S CM5 chip by amine coupling, his-tagged cynomolgus monkey CD40 were captured by the immobilized anti-his antibody with a flow rate of 10 µL min⁻¹ for 60 s. Two-fold serially diluted CD40 antibodies were injected through flow cells for 120 s followed by a 130 s dissociation phase at a flow rate of 30 µL min⁻¹. Prior to next assay cycle, the sensor surface was regenerated with Glycine-HCl (pH 1.5) for 30 s at a flow rate of 30 µL min⁻¹. Background binding to blank immobilized flow cells was subtracted, and KD values were calculated using the 1:1 binding kinetics model built in the BIAcore T200 Evaluation Software (version 3.2).

### CD40 Antibody Selectivity

Human CD40 (Acrobiosystems), GITR (Acrobiosystems), OX40 (Acrobiosystems), 4-1BB (Acrobiosystems) or BSA (Solarbio) were plated onto a microtiter plate at 4° C. overnight. The coated wells were blocked by 0.5% BSA in PBS at 37° C. for 1 hr. Serially diluted antibodies were added and incubated at 37° C. for 1 hr, washed 8 times, before goat anti-human IgG-HRP (SouthernBiotech) was added and incubated at 37° C. for 30 min. After 8 times of washes, ABTS substrate solution (Thermofisher) was added and the OD at 405 nm were measured with a plate reader.

### Jurkat / NF- ĸB-GFP-hCD40 Reporter Cell Assays

For CD40 agonists activity detection, Jurkat / NF-κB-GFP-hCD40 reporter cells were incubated with different concentrations of CD40 agonist antibodies with or without goat anti-human Fc antibody (SouthernBiotech) for 24 hours. GFP expression was detected by flow cytometry.

For FcγRIIB dependency experiment, HEK293T cells were transiently transfected with FcγRIIB expressing vector using PEI. After 36 hours, HEK293T-FcγRIIB cells were plated in 48-well plate and cultured overnight at 37° C. Then Jurkat / NF-κB-GFP-hCD40 reporter cells and different concentrations of C03 or HEL were cocultured with HEK293FT-FcγRIIB cells for 24 hours. GFP expression was detected by flow cytometry.

For data analysis of the Jurkat / NF-κB-GFP-hCD40 reporter cell assays, flow cytometry results were analyzed with software Flowjo X, fluorescence intensity equals to the percentage of GFP positive cells mutiplied by MFI. Fluorescent intensity of the cells was plotted against antibody concentrations calculated using software GraphPad Prism.

### CD40 Ex Vivo Experiment

Following thawing and recovery of human PBMCs, monocytes were selected by adhering to plastic and then cultured for 8 days in RPMI containing 10% FBS (Gibco), 100 ng/mL GM-CSF (R&D Systems) and 10 ng/mL IL-4 (R&D Systems). Suspended cells were harvested and confirmed to be dendritic cells by CD11c expression. B cells were isolated from PBMCs by magnetic selection using CD19 beads (Miltenyi). 1 × 10⁵ dendritic cells or B cells were incubated with different concentrations of C03 with or without goat anti-human Fc antibody(SouthernBiotech) for 48 hours. Upregulation of the activation markers CD86 was analyzed by flow cytometry(Biolegend). Flow cytometry results were analyzed with software Flowjo X. The MFI of cells was plotted against the antibody concentrations using software GraphPad Prism.

### CD40 In Vivo Experiment

All animal experimental procedures were conducted in accordance with the relevant governmental / ethical guides for the care and use of laboratory animals, and were performed according to the institutional ethical guidelines for animal experiment. All experimental procedures were approved by the relevant ethics committee and/or regulatory authorities. All mice were housed under SPF condition.

### OVA-Specific CD8⁺ T-Cell Response Model

CD40/FcγR humanized mice were adoptively transferred with CD45.1⁺ splenic OT-I cells (2 × 10⁶ cells in 200 µl PBS per mouse) via tail vein injection one day before immunized with 2 µg of DEC-OVA, in the presence of the CD40 agonist antibody or the isotype control by intraperitoneal injection. On day 6, spleen cells were harvested. After red blood cells lysis, the single-cell suspension was stained with anti-CD4 (clone RM4-5), anti-CD8 (clone 53-6.7), anti-CD45.1 (A20), anti-TCR-Vα2 (B20.1) to quantify OVA-specific OT-ICD8⁺ T cells. OT-ICD8⁺ T cell is defined as CD45.1⁺CD8⁺ TCR-Vα2⁺ cells.

### Syngeneic Mouse Model

CD40/FcγR humanized mice were inoculated subcutaneously with 2× 10⁶ MC38 cells. When tumor volumes reached 50 to 100 mm³, mice were randomly assigned to different groups (n=5). MC38-bearing CD40/FcγR humanized mice were treated intraperitoneally with C03, CP870893 or HEL (3 mg/kg, q3d×2). Tumor growth was monitored every 3 days by measuring the length (L) and width (W) with calipers and tumor volume was calculated with the formula, (L×W²)/2.

### REFERENCES

1. L. P. Andrews, H. Yano, D. A. A. Vignali, Inhibitory receptors and ligands beyond PD-1, PD-L1 and CTLA-4: breakthroughs or backups. Nature immunology 20, 1425 (Nov, 2019).

2. J. Tang et al., Trial watch: The clinical trial landscape for PD1/PDL1 immune checkpoint inhibitors. Nature reviews. Drug discovery 17, 854 (Nov. 28, 2018).

3. A. Hoos, Development of immuno-oncology drugs - from CTLA4 to PD1 to the next generations. Nature reviews. Drug discovery 15, 235 (Apr, 2016).

4. P. A. Mayes, K. W. Hance, A. Hoos, The promise and challenges of immune agonist antibody development in cancer. Nature reviews. Drug discovery 17, 509 (Jul, 2018).

5. R. H. Vonderheide, CD40 Agonist Antibodies in Cancer Immunotherapy. Annual review of medicine 71, 47 (Jan. 27, 2020).

6. A. F. Labrijn, M. L. Janmaat, J. M. Reichert, P. Parren, Bispecific antibodies: a mechanistic review of the pipeline. Nature reviews. Drug discovery 18, 585 (Aug, 2019).

7. H. Li, P. Er Saw, E. Song, Challenges and strategies for next-generation bispecific antibody-based antitumor therapeutics. Cellular & molecular immunology 17, 451 (May, 2020).

8. G P. Smith, Filamentous fusion phage: novel expression vectors that display cloned antigens on the virion surface. Science 228, 1315 (Jun. 14, 1985).

9. R. W. Roberts, J. W. Szostak, RNA-peptide fusions for the in vitro selection of peptides and proteins. Proceedings of the National Academy of Sciences of the United States of America 94, 12297 (Nov. 11, 1997).

10. J. McCafferty, A. D. Griffiths, G Winter, D. J. Chiswell, Phage antibodies: filamentous phage displaying antibody variable domains. Nature 348, 552 (Dec. 6, 1990).

11. R. A. Lerner, Manufacturing immunity to disease in a test tube: the magic bullet realized. Angew Chem Int Ed Engl 45, 8106 (Dec. 11, 2006).

12. W. D. Huse et al., Generation of a large combinatorial library of the immunoglobulin repertoire in phage lambda. Science 246, 1275 (Dec. 8, 1989).

13. C. F. Barbas, 3rd, A. S. Kang, R. A. Lerner, S. J. Benkovic, Assembly of combinatorial antibody libraries on phage surfaces: the gene III site. Proceedings of the National Academy of Sciences of the United States of America 88, 7978 (Sep. 15, 1991).

14. H. Zhang et al., Autocrine selection of a GLP-1R G-protein biased agonist with potent antidiabetic effects. Nat Commun 6, 8918 (Dec. 1, 2015).

15. H. Zhang, I. A. Wilson, R. A. Lerner, Selection of antibodies that regulate phenotype from intracellular combinatorial antibody libraries. Proceedings of the National Academy of Sciences of the United States of America 109, 15728 (Sep. 25, 2012).

16. S. L. Anna., N. Bontoux., H. A. Stone., Formation of dispersions using “flow focusing” in microchannels. Applied Physics Letters 82, 364 (2003).

17. L. Mazutis et al., Single-cell analysis and sorting using droplet-based microfluidics. Nat Protoc 8, 870 (May, 2013).

18. N. Shembekar, C. Chaipan, R. Utharala, C. A. Merten, Droplet-based microfluidics in drug discovery, transcriptomics and high-throughput molecular genetics. Lab Chip 16, 1314 (Apr. 21, 2016).

19. G Georgiou et al., The promise and challenge of high-throughput sequencing of the antibody repertoire. Nat Biotechnol 32, 158 (Feb, 2014).

20. B. El Debs, R. Utharala, I. V. Balyasnikova, A. D. Griffiths, C. A. Merten, Functional single-cell hybridoma screening using droplet-based microfluidics. Proceedings of the National Academy of Sciences of the United States of America 109, 11570 (Jul. 17, 2012).

21. N. Shembekar, H. Hu, D. Eustace, C. A. Merten, Single-Cell Droplet Microfluidic Screening for Antibodies Specifically Binding to Target Cells. Cell reports 22, 2206 (Feb. 20, 2018).

22. K. Eyer, R. C. L. Doineau, C. E. Castrillon, L. Briseño-Roa, Single-cell deep phenotyping of IgG-secreting cells for high-resolution immune monitoring. Nat Biotechnol 35, 977 (Oct, 2017).

23. A. Gérard, A. Woolfe, G Mottet, M. Reichen, C. Castrillon, High-throughput single-cell activity-based screening and sequencing of antibodies using droplet microfluidics. Nat Biotechnol 38, 715 (Jun, 2020).

24. L. Schmid, D. A. Weitz, T. Franke, Sorting drops and cells with acoustics: acoustic microfluidic fluorescence-activated cell sorter. Lab Chip 14, 3710 (Oct. 7, 2014).

25. R. Lutterbuese et al., T cell-engaging BiTE antibodies specific for EGFR potently eliminate KRAS- and BRAF-mutated colorectal cancer cells. Proceedings of the National Academy of Sciences of the United States of America 107, 12605 (Jul. 13, 2010).

26. M. Chodorge et al., A series of Fas receptor agonist antibodies that demonstrate an inverse correlation between affinity and potency. Cell death and differentiation 19, 1187 (Jul, 2012).

27. T. Kula et al., T-Scan: A Genome-wide Method for the Systematic Discovery of T Cell Epitopes. Cell 178, 1016 (Aug. 8, 2019).

28. J. B. Spangler, I. Moraga, J. L. Mendoza, K. C. Garcia, Insights into cytokine-receptor interactions from cytokine engineering. Annual review of immunology 33, 139 (2015).

29. J. T. Sockolosky et al., Selective targeting of engineered T cells using orthogonal IL-2 cytokine-receptor complexes. Science 359, 1037 (Mar. 2, 2018).

30. P. N. Kelly, Engineering cytokine-receptor pairs. Science 359, 1004 (2018).

31. J. C. McDonald, G M. Whitesides, Poly(dimethylsiloxane) as a material for fabricating microfluidic devices. Acc Chem Res 35, 491 (Jul, 2002).

All cited references are incorporated herein by reference in the place of citation.

1. A method of identifying an agonist or antagonist polypeptide of a biological function, the method comprising:
(1) providing a plurality of nano- or pico-liter droplets, each comprising:
(i) no more than one library cell that (if present) expresses or is capable of expressing a candidate agonist or antagonist polypeptide from a library of candidate agonist or antagonist polypeptides;
(ii) a reporter cell that, upon contacting the agonist or antagonist polypeptide of the biological function, produces a detectable signal as a marker or indicative of said biological function;
(2) maintaining the plurality of nano- or pico-liter droplets under a suitable condition to permit said agonist or antagonist polypeptide to contact the report cell to trigger the biological function, thereby producing said detectable signal;
(3) isolating or enriching nano- or pico-liter droplets manifesting said detectable signal,
thereby identifying the agonist or antagonist polypeptide of said biological function, within the isolated or enriched nano- or pico-liter droplets.

2. The method of claim 1, wherein expression of the candidate agonist or antagonist polypeptide from the library is under the control of an inducible promoter inducible by an activator or an activating condition.

3. The method of claim 2, wherein the inducible promoter is a positive inducible promoter, and wherein an activator for said positive inducible promoter is introduced into said plurality of nano- or pico-liter droplets subsequent to the formation of said plurality of nano- or pico-liter droplets.

4. The method of claim 3, wherien:
(1) the positive inducible promoter is a Tet-ON promoter, and wherein the activator is tetracycline or a derivative thereof capable of binding to activator rtTA (reverse tetracycline-controlled transactivator);
(2) the positive inducible promoter is an alcohol-regulated promoter (such as the AlcA promoter), and wherein the activator is AlcR or AlcA; or,
(3) the positive inducible promoter is a steroid-regulated promoter (such as the LexA promoter), and wherein the activator is XVE.

5. The method of claim 2, wherein the inducible promoter is a negative inducible promoter, and wherein an activator for said negative inducible promoter is introduced into said plurality of nano- or pico-liter droplets subsequent to the formation said plurality of nano- or pico-liter droplets.

6. The method of claim 5, wherien:
(1) the negative inducible promoter is a pLac promoter, and wherein the activator is lactose or a derivative thereof (such as IPTG) capable of binding to lac repressor (lacI protein); or,
(2) the negative inducible promoter is a pBad promoter, and wherein the activator is arabinose capable of binding to AraC.

7. The method of claim 2, wherein the inducible promoter is a temperature sensitive promoter, and the expression of the candidate agonist or antagonist polypeptide from the library is under the control of a temperature change as the activating condition that activates the inducible promoter.

8. The method of claim 2, wherein the inducible promoter is a light inducible promoter (such as the FixK2 promoter), and the expression of the candidate agonist or antagonist polypeptide from the library is under the control of a light signal as the activating condition that activates the light inducible promoter.

9. The method of any one of claims 2-6, wherein the activator is introduced into said plurality of nano- or pico-liter droplets via injection into said plurality of nano- or pico-liter droplets, or via fusion.

10. The method of claim 9, wherein said fusion is mediated by geometrical constraint, mechanical force, surface property change, electrical, laser, or acoustic force.

11. The method of claim 1, wherein prior to step (1), a first plurality of nano- or pico-liter droplets each comprising said no more than one library cell have been maintained under a pre-determined condition for a pre-determined period of time to allow said candidate agonist or antagonist polypeptide to express, before said reporter cell is introduced into each said first plurality of nano- or pico-liter droplets to provide the plurality of nano- or pico-liter droplets in step (1).

12. The method of claim 11, wherein the reporter cell is introduced via injection or via fusion.

13. The method of claim 12, wherein said fusion is mediated by geometrical constraint, mechanical force, surface property change, electrical, laser, or acoustic force.

14. The method of any one of claims 1-13, wherein said library is a library of expression vectors, such as lentiviral vector, retroviral vector, sindbis viral vector, or plasmid.

15. The method of any one of claims 1-14, wherein said library cell is a cell from a tumor cell line, a T cell line, or a NK cell line.

16. The method of any one of claims 1-15, wherein steps (1)-(3) are repeated more than once using said library cell isolated or enriched in step (3) of a previous repeat.

17. The method of any one of claims 1-16, wherein said plurality of nano- or pico-liter droplets each comprises a 3^{rd} cell that facilitates the production of the detectable signal.

18. The method of any one of claims 1-17, wherein said library cell that expresses or is capable of expressing a candidate agonist or antagonist polypeptide is pre-stained with a first tracking signal (e.g., CellTrace Violet), and said reporter cell is pre-stained with a second, different, tracking signal (e.g., Cell Trace Yellow) prior to step (1), and wherein step (3) is carried out by retrieving nano- or pico-liter droplets that:
(I) contain both the first and the second tracking signals;
(II) produce said detectable signal (e.g., GFP) after step (2); and,
(III) exhibit colocalization of the second (reporter cell) tracking signal and the detectable signal.

19. The method of any one of claims 1-18, wherein the agonist or antagonist polypeptide is an antibody, a bispecific antibody, a tri-specific antibody, or an antigen-binding fragment thereof (including antibodies or antigen-binding fragment thereof having similar CDR sequence except for random mutations in the CDR sequences for affinity maturation), a polypeptide, a cytokine, a chemokine, or a derivative thereof.

20. The method of any one of claims 1-19, wherein the agonist or antagonist polypeptide is a bispecific T cell engager (BiTE) comprising a first antigen-binding fragment (such as a 1^{st} scFv) specific for a first antigen fused to a second antigen-binding fragment (such as a 2^{nd} scFv) specific for a second antigen.

21. The method of claim 20, wherein the first antigen is a T cell antigen (such as CD3), and the second antigen is a surface antigen on a target cell (such as a cancer antigen (e.g., HER2) on a target cancer cell).

22. The method of claim 21, wherein in each nano- or pico-liter droplet having said one cell that expresses or is capable of expressing said candidate agonist or antagonist polypeptide, said candidate agonist or antagonist polypeptide is a BiTE from a library of candidate BiTEs each encoded by a lentiviral vector from a lentiviral vector library encoding said library of candidate BiTEs, and wherein said one cell is the target cell that expresses said target cancer antigen (e.g., HER2).

23. The method of claim 22, wherein the reporter cell is a T cell-derived cell line (e.g., Jurkat cell) that produces a fluorescent protein (e.g., GFP), the transcription of which encoding RNA is under the transcriptional control of a promoter (e.g., IL-2 promoter) activated by T cell activation upon binding of the BiTE to the TCR of the reporter cell and the target cancer antigen on the target cell.

24. The method of any one of claims 20-23, wherein said library of candidate agonist or antagonist polypeptides is a library of candidate BiTEs encoded by a lentiviral vector-based library, and wherein coding sequence for each of said second antigen-binding fragment (such as a 2^{nd} scFv) specific for the second antigen has been pre-selected from a phage display library based on biopanning against said second antigen.

25. The method of claim 24, wherein the complexity of the phage display library is about 10¹⁰ members, and wherein the complexity of the library of candidate BiTEs with respect to the second antigen-binding fragment is 10⁵ members.

26. The method of any one of claims 20-25, wherein said one cell that expresses or is capable of expressing the candidate agonist or antagonist polypeptide (BiTE) is produced by infection at low MOI, by a lentiviral vector-based library encoding said library of candidate agonist or antagonist polypeptides (BiTEs), to ensure that each cell produces no more than one type of the candidate agonist or antagonist polypeptide (BiTE).

27. The method of any one of claim 1-19, wherein the agonist or antagonist polypeptide is an agonist or antagonist antibody or an antigen-binding fragment thereof specific for a cell surface receptor (e.g., CD40) that triggers said biological function.

28. The method of claim 27, wherein in each nano- or pico-liter droplet having said one cell that expresses or is capable of expressing said candidate agonist or antagonist polypeptide, said candidate agonist or antagonist polypeptide is an scFv-IgG1 Fc fusion from a library of candidate scFv-IgG1 Fc fusions each encoded by a lentiviral vector from a lentiviral vector library encoding said library of candidate scFv-IgG1 Fc fusions, optionally, wherein said cell surface receptor is CD40 and wherein said biological function is NFκB signaling.

29. The method of claim 28, wherein the reporter cell is a cell line (e.g., Jurkat cell) that produces a fluorescent protein (e.g., GFP), the transcription of which encoding RNA is under the transcriptional control of a promoter (e.g., NFκB promoter) activated by activation of said cell surface receptor (e.g., CD40) upon binding of the agonist antibody or antigen-binding fragment thereof to the cell surface receptor (e.g., CD40) of the reporter cell.

30. The method of any one of claims 27-29, wherein coding sequence for each of said scFv in said library of candidate scFv-IgG1 Fc fusions has been pre-selected from a phage display library based on biopanning against said cell surface receptor (e.g., CD40).

31. The method of claim 30, wherein the complexity of the phage display library is about 10¹⁰ members, and wherein the complexity of the library of candidate scFv-IgG1 Fc fusions with respect to the second antigen-binding fragment is 10⁵ members.

32. The method of any one of claims 27-31, wherein said one cell that expresses or is capable of expressing the candidate agonist or antagonist polypeptide is produced by infection at low MOI, by a lentiviral vector-based library encoding said library of candidate agonist or antagonist polypeptides, to ensure that each cell produces no more than one type of the candidate agonist or antagonist polypeptide.

33. The method of any one of claims 27-32, wherein a secondary antibody specific for said candidate agonist or antagonist polypeptide is labeled with a first tracking signal (e.g., Dylight647-conjugated) and co-encapsulated into the nano- or pico-liter droplets in step (1), and said reporter cell is pre-stained with a second, different, tracking signal (e.g., Cell Trace Yellow) prior to step (1), and wherein step (3) is carried out by retrieving nano- or pico-liter droplets that:
(I) contain both the first and the second tracking signals;
(II) produce said detectable signal (e.g., GFP) after step (2); and,
(III) exhibit colocalization of the first (CD40 agonist antibodies) and the second (reporter cell) tracking signals and the detectable signal.

34. The method of any one of claims 1-19, wherein the agonist or antagonist polypeptide is an engineered or modified cytokine for a cytokine receptor that triggers said biological function.

35. The method of claim 34, wherein the engineered or modified cytokine has altered specificity and/or affinity towards the cytokine receptor compared to the cognate wild-type cytokine.

36. The method of claim 34, wherein the engineered or modified cytokine binds to and activates a cytokine receptor to which a cognate wild-type cytokine does not bind.

37. The method of claim 34, wherein the engineered or modified cytokine stimulates or inhibits a downstream signaling pathway that is not stimulated by a cognate wild-type cytokine.

38. The method of claim 34, wherein the engineered or modified cytokine commits a cell to a differentiation, proliferation, activation, and/or apoptotic process that is not stimulated or inhibited by a cognate wild-type cytokine, or is not stimulated or inhibited by the cognate wild-type cytokine to the same degree.

39. The method of any one of claims 1-38, wherein step (1) is carried out with a nano- or pico-liter droplet-producing microfluidic device comprising:
(a) a first inlet for an oil to form a continuous oil phase;
(b) a second inlet for an aqueous suspension of a population of said reporter cell;
(c) a third inlet for an aqueous suspension of a population of said cell that expresses or is capable of expressing a candidate agonist polypeptide;
(d) an outlet for retrieving said nano- or pico-liter droplets dispersed in said continuous oil phase; and,
(e) a junction area where the first, the second, and the third inlets converge to form nano- or pico-liter droplets in the continuous oil phase before exiting through the outlet.

40. The method of claim 39, wherein step (3) is carried out in a nano- or pico-liter droplet-sorting microfluidic device comprising:
(A) a first inlet of spacing oil and a second inlet of bias oil;
(B) a third inlet of retrieved nano- or pico-liter droplets after step (2);
(C) a first outlet for retrieving nano- or pico-liter droplets manifesting said detectable signal;
(D) a second outlet for collecting waste not retrieved by the first outlet;
(E) a sorting actuator that directs a passing nano- or pico-liter droplet to the first outlet when the passing nano- or pico-liter droplet manifests the detectable signal, and directs the passing nano- or pico-liter droplet to the second outlet otherwise; and,
(F) a junction area where the first, the second, and the third inlets converge to form a stream of passing nano- or pico-liter droplets before the sorting actuator, and where the first and second outlets diverge to separate said nano- or pico-liter droplets manifesting said detectable signal from the waste.

41. The method of any one of claims 1-40, wherein said agonist or antagonist polypeptide is identified through identifying the coding sequence thereof from said cell that expresses or is capable of expressing said agonist or antagonist polypeptide retrieved from said nano- or pico-liter droplets manifesting said detectable signal.

42. The method of claim 41, further comprising verifying that said agonist or antagonist polypeptide leads to said biological function, including activation of said biological function by said agonist or antagonist polypeptide in a manner depending on binding by said agonist or antagonist polypeptide.
